(19) United States
(12) Patent Application Publication
(10) Pub. No.: US 2025/0170363 A1
(43) Pub. Date: May 29, 2025

(54) ROBOTIC CATHETER TIP AND METHODS AND STORAGE MEDIUMS FOR CONTROLLING AND/OR MANUFACTURING A CATHETER HAVING A TIP

(71) Applicant: CANON U.S.A., INC., Melville, NY (US)

(72) Inventors: Emma Lynn Hawkes, Somerville, MA (US); Benedict Shia, Needham, MA (US); Takahisa Kato, Brookline, MA (US); Albert Harold Dunfee, Byfield, MA (US); Inderpal Singh, Norwood, MA (US)

(21) Appl. No.: 18/943,386

(22) Filed: Nov. 11, 2024

Related U.S. Application Data
(60) Provisional application No. 63/603,006, filed on Nov. 27, 2023.

(51) Int. Cl. A61M 25/00; A61B 34/30

(52) U.S. Cl. CPC A61M 25/001; A61B 2034/301

(57) ABSTRACT

One or more devices, systems, methods, and storage mediums for performing robotic control and/or for manufacturing and/or using a catheter having a tip are provided herein. Examples of such control, catheter manufacturing and use include, but are not limited to, correction of one or more sections or portions of a continuum robot as the continuum robot is moved. Examples of applications include imaging, evaluating, and diagnosing biological objects, such as, but not limited to, for bronchial or other medical applications, and being obtained via one or more optical instruments, such as, but not limited to, optical probes, catheters, endoscopes, and bronchoscopes. Techniques provided herein also improve processing, imaging, and catheter control efficiency while achieving images that are more precise, and also achieve devices, systems, methods, and storage mediums that reduce mental and physical burden and improve ease of use.

### CROSS-REFERENCE TO RELATED APPLICATION(S)

This application relates, and claims priority, to U.S. Prov. Patent Application Ser. No. 63/603,006, filed Nov. 27, 2023, the disclosure of which is incorporated by reference herein in its entirety.

### FIELD OF THE DISCLOSURE

The present disclosure generally relates to imaging and, more particularly, to an apparatus, method, and storage medium for making, and/or use with, a robotic catheter tip and/or for implementing robotic control for all sections of a catheter or imaging device/apparatus or system to match a state or states when each section reaches or approaches a same or similar, or approximately a same or similar, state or states of a first section of the catheter or imaging device, apparatus, or system. The present disclosure generally relates to imaging and, more particularly, to bronchoscope(s), robotic bronchoscope(s), robot apparatus(es), method(s), and storage medium(s) that operate to image a target, object, or specimen (such as, but not limited to, a lung, a biological object or sample, tissue, etc.). One or more bronchoscopic, endoscopic, medical, camera, catheter, or imaging devices, systems, and methods and/or storage mediums for use with same, are discussed herein. One or more devices, methods, or storage mediums may be used for medical applications and, more particularly, to steerable, flexible medical devices that may be used for or with guide tools and devices in medical procedures, including, but not limited to, bronchoscopes, endoscopes, cameras, and catheters.

### BACKGROUND

Medical imaging is used with equipment to diagnose and treat medical conditions. Endoscopy, bronchoscopy, catheterization, and other medical procedures facilitate the ability to look inside a body. During such a procedure, a flexible medical tool may be inserted into a patient's body, and an instrument may be passed through the tool to examine or treat an area inside the body. For example, a scope can be used with an imaging device that views and/or captures objects or areas. The imaging can be transmitted or transferred to a display for review or analysis by an operator, such as a physician, clinician, technician, medical practitioner or the like. The scope can be an endoscope, bronchoscope, or other type of scope. By way of another example, a bronchoscope is an endoscopic instrument to look or view inside, or image, the airways in a lung or lungs of a patient. The bronchoscope may be put in the nose or mouth and moved down the throat and windpipe, and into the airways, where views or imaging may be made of the bronchi, bronchioles, larynx, trachea, windpipe, or other areas.

Catheters and other medical tools may be inserted through a tool channel in the bronchoscope to provide a pathway to a target area in the patient for diagnosis, planning, medical procedure(s), treatment, etc.

Robotic bronchoscopes, robotic endoscopes, or other robotic imaging devices may be equipped with a tool channel or a camera and biopsy tools, and such devices (or users of such devices) may insert/retract the camera and biopsy tools to exchange such components. The robotic bronchoscopes, endoscopes, or other imaging devices may be used in association with a display system and a control system.

An imaging device, such as a camera, may be placed in the bronchoscope, the endoscope, or other imaging device/system to capture images inside the patient and to help control and move the bronchoscope, the endoscope, or the other type of imaging device, and a display or monitor may be used to view the captured images. An endoscopic camera that may be used for control may be positioned at a distal part of a catheter or probe (e.g., at a tip section).

The display system may display, on the monitor, an image or images captured by the camera, and the display system may have a display coordinate used for displaying the captured image or images. In addition, the control system may control a moving direction of the tool channel or the camera. For example, the tool channel or the camera may be bent according to a control by the control system. The control system may have an operational controller (such as, but not limited to, a joystick, a gamepad, a controller, an input device, etc.), and physicians may rotate or otherwise move the camera, probe, catheter, etc. to control same. However, such control methods or systems are limited in effectiveness. Indeed, while information obtained from an endoscopic camera at a distal end or tip section may help decide which way to move the distal end or tip section, such information does not provide details on how the other bending sections or portions of the bronchoscope, endoscope, or other type of imaging device may move to best assist the navigation.

However, while a camera may provide information for how to control a most distal part of a catheter or a tip of the catheter, the information is limited in that the information does not provide details about how the other bending sections of the catheter or probe should move to best assist the navigation.

While previously technology may attach a polymer on a passive braided shaft through a thermal process, with heat shrink and multiple layers of polymer in order to contain any free wire ends of the braided shaft while forming an atraumatic tip. Typically, tip design accommodates a sensor, such as an Electromagnetic (EM) sensor, and provides an atraumatic distal tip for navigating through anatomy.

Despite previous technology providing an atraumatic tip, the previous technology fails to address any needs for maintaining and/or forming specific internal geometry and/or structure of the tip, while also protecting proximal features on a catheter that are important to the function(s) of the catheter (which leaves previous technology susceptible or vulnerable to heat and/or damage. Previous technology uses a multi-step and multi-piece tip design that requires many manufacturing process steps, consumable materials, and manufacturing time to integrate into a distal end of a catheter, which drives up the overall cost of the catheter. Since such manufacturing methods (and structure resulting from same) are also inconsistent, repair and maintenance of the previous catheter technology is costly. Navigation performance of previous technology is also reduced due to the use of rigid, non-reflowable material, which is only able to be integrated through adhesive processes, and the multi-piece design with adhesive processes creates a longer rigid length on a distal end of the catheter than is needed, which negatively impacts navigation performance.

As such, there is a need for devices, systems, methods, and/or storage mediums that address the above issues by providing rapid, accurate, cost-effective, and minimally invasive structure and manufacture/use techniques for structure of a catheter and/or a catheter tip.

### SUMMARY

Accordingly, it is a broad object of the present disclosure to provide advantageous features to imaging devices, apparatuses, systems, methods, and/or storage mediums, such as, but not limited to, using robotic and/or catheter features, by providing consistent manufacture/use techniques (and resulting structure from same) and by providing rapid, accurate, cost-effective, consistent, and minimally invasive structure and manufacture/use techniques for structure of a catheter and/or a catheter tip.

A robotic catheter and/or autonomous robot may be used in one or more embodiments of the present disclosure to address the above issues, and robotic catheter and/or autonomous robot apparatuses, systems, methods, storage mediums, and/or other related features may be used to increase maneuverability into an object, sample, or target (e.g., a patient, an organ of a patient, a tissue being imaged or evaluated, a lung, a lumen, a vessel, another part of a patient, etc.), while preserving visualization and catheter stability.

Additionally, it is another broad object of the present disclosure to provide imaging (e.g., computed tomography (CT), Magnetic Resonance Imaging (MRI), etc.) apparatuses, systems, methods, and storage mediums for using a navigation and/or control method or methods (manual or automatic) in one or more apparatuses or systems (e.g., an imaging apparatus or system, a catheter, an autonomous catheter or robot, an endoscopic imaging device or system, etc.).

In one or more embodiments, an apparatus may include one or more controllers and/or one or more processors, the controller(s) and/or processor(s) operating to perform advancing a medical tool and/or an imaging tool (e.g., a camera) through a lumen and/or pathway.

In one or more embodiments of the present disclosure, an apparatus may include: a catheter, and an atraumatic tip attached to the distal portion of the catheter. In one or more embodiments, the atraumatic tip may be thermally attached to the distal portion of the catheter. The atraumatic tip may be designed to include all critical geometry and/or structure related to catheter function, and the geometry and/or structure may operate to be preserved through a manufacturing process(es). In one or more embodiments, the atraumatic tip may be a piece molded from a reflowable polymer material. The atraumatic tip may have a counterbore that operates to accommodate a capture piece and/or a distal end of a braided shaft. Additional geometry for alignment or continuity may be added as needed. For example, additional extruded bosses on a proximal side of the atraumatic tip may key into multi-lumens on a drive ring anchor of the catheter. The atraumatic tip may have a durometer, and, while not limited thereto, the durometer may be one of the following: 35 D, 55 D, 63 D, 72 D, and/or a value in a range of 35 D-72 D. In one or more embodiments, the atraumatic tip may be radiopaque, and the atraumatic tip may sit flush with a distal drive ring of the apparatus.

The atraumatic tip may include a tip piece and a capture piece, where the tip piece may be attached to the capture piece that operates to capture (or fully capture) a braided inner cover or shaft of the catheter. The capture piece may be made of a reflowable polymer material, compatible with the braided inner cover or shaft. The polymer material for the capture piece may be Pebax (or any other block copolymer variation of PEBA (polyether block amide)). The capture piece may be made of higher durometer than the atraumatic tip piece so that the higher durometer may provide useful structure for the point and may preserve the captured ends of the braided inner cover or shaft during later reflow processes of the tip piece of the atraumatic tip. In one or more embodiments, the capture piece may be, or may also be, radiopaque. RF stripes, radiopaque stripes, or another alternative color stripe may be useful for indicating the orientation of the atraumatic tip and capture during the attachment process. The colored stripes may also aid in camera loading and docking into the anti-twist feature on a cap of the atraumatic tip. RF stripes or radiopaque stripes may be custom designed for this or any other purpose or feature discussed herein. The capture piece operates to allow free wire ends of the braided inner cover or shaft to be fully secured and anchored within a layer of polymer prior to being integrated into the tip piece of the atraumatic tip. The capture piece on the braided inner cover or shaft may be seated into the tip piece of the atraumatic tip. The atraumatic tip piece may be attached flush against the last drive or guide ring of the catheter, and/or the atraumatic tip piece may be joined and flown together with the last distal drive ring or guide through the thermal attachment process(es) or step(s).

In one or more embodiments of the present disclosure, an apparatus may include: a catheter having at least a braided inner cover or shaft, a capture piece attached to a distal end of the braided inner cover or shaft, and an atraumatic tip piece attached with or to a distal end of the capture piece. In one or more embodiments, the atraumatic tip piece may bridge the capture piece to the braided inner cover or shaft (e.g., such that the atraumatic tip piece may touch or contact the braided inner cover or shaft). The atraumatic tip piece may include a counterbore to accommodate/bridge the capture piece and the braided inner cover or shaft. The atraumatic tip piece may include a tool channel, and the tool channel may be connected to the inner diameter of the braided inner cover or shaft continuously (e.g., the tool channel may be co-axial with the inner diameter of the braided inner cover or shaft, the tool channel may be in alignment with the inner diameter of the braided inner cover or shaft, the inner diameter of the braided inner cover or shaft may match or substantially match the diameter of the tool channel of the atraumatic tip piece, etc.). As described above, the capture piece and/or the atraumatic tip piece may be made of polymer, thermoplastic elastomer, or other similar material known to those skilled in the art. As aforementioned, the capture piece and/or the atraumatic piece may be made of a radiopaque material.

The drive or guide ring(s) may be attached to the braided inner cover or shaft. Driving wires may be passively threaded through the lumens of the guide or drive ring(s) and may be terminated at a distal most guide or drive ring. The distal most guide or drive ring may be attached with or to the atraumatic tip piece. In one or more embodiments, the atraumatic tip piece may include one or more extruded or extruding bosses that are inserted to, or operate to be inserted to, one or more respective acceptors in the distal most guide or drive ring. The atraumatic tip piece may include a tool channel having or including an orientation feature where a camera may be detachably attached with, or removably inserted, into the tool channel. The orientation feature may determine a rotational orientation of the camera to the atraumatic tip piece, and the extruded bosses may determine a rotational orientation of the atraumatic tip to the distal most guide or drive ring.

In one or more embodiments, the catheter may include a tracking sensor that operates to measure a position and orientation (or other state information) of the distal end of the catheter. The tracking sensor may be attached to or disposed in the atraumatic tip piece. The tracking sensor may operate to locate an area from the distal end of the atraumatic tip piece to the capture piece (e.g., along a longitudinal direction, along a longitudinal axis of the catheter, in a direction co-linear or co-axial with the tool channel of the atraumatic tip piece and/or the inner diameter of the braided inner cover or shaft, etc.).

In one or more embodiments of the present disclosure, a method of making a robotic catheter or an imaging apparatus may include: attaching a distal end of a braided inner cover or shaft of a catheter with or to a capture piece, and attaching an atraumatic tip piece to a distal end of the capture piece. The attachment steps may include or involve a thermal attachment process where at least the atraumatic tip piece is thermally attached to the catheter. The thermal attachment process may provide: a stronger overall joint than adhesive processes, a continuous inner lumen of the catheter, and/or all free wire ends of the braided inner cover or shaft may be fully captured and seated within the reflowed polymer (without risk of penetration to the inner lumen of the catheter). In one or more embodiments, the method may further include selecting a softer durometer material for a molded atraumatic tip piece as compared to the hardness of the capture piece to provide a configuration where: (i) the capture piece on the braided inner cover or shaft may remain intact while the molded atraumatic tip piece may be seated around the capture piece, (ii) free wire ends of the braided inner cover or shaft may not penetrate, or avoid penetration with, the inner or outer diameter of the catheter, and (iii) the wire ends are securely disposed or placed within a joint or bridge of the atraumatic tip piece that extends over and surrounds the capture piece of the catheter. In one or more embodiments, the method may further include applying or including radiopaque stripes, RF stripes, or other color stripes as visual aids on the catheter. RF stripes may be used for visualization under fluoroscopy (for example, while the apparatus or catheter is in use). Using added colorants or stripes may provide an advantage for visualization as the camera is loaded and passed through the inner lumen of the catheter. For example, use of colorants or stripes may allow for docking of the camera to an anti-twist feature in the atraumatic tip piece more seamlessly. In one or more embodiments, the method(s) may further include using a specific, set, or predetermined internal geometry and/or structure for the molded piece for the atraumatic tip piece so that the catheter remains intact, and the specific, set, or predetermined internal geometry and/or structure may include one or more of the following: (i) geometry and/or structure that operates to maintain or improve flow and vacuum suction rates; and/or (ii) geometry and/or structure that operates to achieve docking of the camera within the catheter. The method may further include using or adding extruded or extruding bosses on a proximal side of the atraumatic tip piece to align to the proximal skeleton or structure of the catheter (e.g., a robotic catheter), which may aid in the manufacturing process and provide consistent tip orientation between individual catheters. The method may further include disposing or incorporating a sensor (e.g., an electromagnetic (EM) sensor) within the geometry and/or structure of the distal tip and/or the atraumatic tip piece. The sensor may be used for tracking, and the EM sensor may be seated (or fully seated) within the distal tip or atraumatic tip piece and be protected by or within the distal tip piece. In one or more embodiments, the method may include fully integrating the sensor or EM sensor into the tip to better maintain or to achieve a smooth outer diameter on the catheter. The aforementioned geometrical and/or structural achievements of the manufacturing processes may be included in and possessed by the geometry and/or structure of any catheter of the present disclosure.

In one or more embodiments of the present disclosure, a storage medium stores instructions or a program for causing one or more processors of an apparatus or system to perform a method of manufacturing a robotic catheter or imaging apparatus, where the method may include: attaching a distal end of a braided inner cover or shaft of a catheter with or to a capture piece, and attaching an atraumatic tip piece to a distal end of the capture piece.

In one or more embodiments, an apparatus for performing navigation control and/or for controlling, manufacturing, or using a catheter and/or catheter tip may include a flexible medical device or tool; and one or more processors that operate to: bend a distal portion of the flexible medical device or tool; and advance the flexible medical device or tool through a pathway, wherein the flexible medical device or tool may be advanced through the pathway in a substantially centered manner.

In one or more embodiments, the flexible medical device or tool may have multiple bending sections, and the one or more processors may further operate to control or command the multiple bending sections of the flexible medical device or tool using one or more of the following modes: a Follow the Leader (FTL) mode, a Reverse Follow the Leader (RIFTL) mode, a Hold the Line mode, a Close the Gap mode, and/or a Stay the Course mode. The flexible medical device or tool may include a catheter or scope and the catheter or scope may be part of, include, or be attached to an imaging apparatus, such as, but not limited to, an endoscope, a catheter, a probe, a bronchoscope, or any other imaging device discussed herein or known to those skilled in the art.

In one or more embodiments, a method for controlling an apparatus including a flexible medical device or tool that operates to perform navigation control and/or for controlling, manufacturing, or using a catheter and/or catheter tip may include: bending a distal portion of the flexible medical device or tool; and advancing the flexible medical device or tool through a pathway, wherein the flexible medical device or tool may be advanced through the pathway in a substantially centered manner. The flexible medical device or tool may have multiple bending sections, and the method may further include controlling or commanding the multiple bending sections of the flexible medical device or tool using one or more of the following modes: a Follow the Leader (FTL) process or mode, a Reserve Follow the Leader (RFTL) process or mode, a Hold the Line process or mode, a Close the Gap process or mode, and/or a Stay the Course process or mode.

In one or more embodiments, a non-transitory computer-readable storage medium storing at least one program for causing a computer to execute a method for controlling an apparatus including a flexible medical device or tool that operates to perform navigation control and/or for controlling, manufacturing, or using a catheter and/or catheter tip, where the method may include: bending a distal portion of the flexible medical device or tool; and advancing the flexible medical device or tool through a pathway, wherein the flexible medical device or tool may be advanced through the pathway in a substantially centered manner. The method may include any other feature discussed herein.

One or more robotic control methods of the present disclosure may be employed in one or more embodiments. For example, one or more of the techniques, modes, or methods may be used as discussed herein, including, but not limited to: Follow the Leader, Reverse Follow the Leader, Hold the Line, Close the Gap, and/or Stay the Course. In one or more embodiments, one or more other or additional robotic control methods or techniques may be employed.

In one or more embodiments, a continuum robot for performing robotic control may include: one or more processors that operate to: instruct or command a first bending section or portion of a catheter or a probe of the continuum robot such that the first bending section or portion achieves, or is disposed at, a pose, position, or state at a position along a path, the catheter or probe of the continuum robot having a plurality of bending sections or portions and a base; instruct or command each of the other bending sections or portions of the plurality of bending sections or portions of the catheter or probe to match, substantially match, or approximately match the pose, position, or state of the first bending section or portion at the position along the path in a case where each section or portion reaches or approaches a same, similar, or approximately similar state or states at the position along the path; and instruct or command the plurality of bending sections or portions such that the first bending section or portion or a Tip or distal bending section or portion is located in a predetermined pose, position, or state at or near a distal end of the path. A first bending section or portion or the Tip or distal bending section or portion may include a camera, an endoscopic camera, a sensor, or other imaging device or system to obtain one or more images of or in a target, sample, or object; and the one or more processors may further operate to command the camera, sensor, or other imaging device or system to obtain the one or more images of or in the target, sample, or object at the predetermined pose, position, or state, and the one or more processors operate to receive the one or more images and/or display the one or more images on a display.

The method(s) may further include any of the features discussed herein that may be used in the one or more apparatuses of the present disclosure.

In one or more embodiments, a non-transitory computer-readable storage medium may store at least one program for causing a computer to execute a method for performing robotic control, and may use any of the method feature(s) discussed herein.

In accordance with one or more embodiments of the present disclosure, apparatuses and systems, and methods and storage mediums for performing navigation, movement, and/or control, and/or for controlling, manufacturing, or using a catheter and/or catheter tip, may operate to characterize biological objects, such as, but not limited to, blood, mucus, tissue, etc.

One or more embodiments of the present disclosure may be used in clinical application(s), such as, but not limited to, intervascular imaging, intravascular imaging, bronchoscopy, atherosclerotic plaque assessment, cardiac stent evaluation, intracoronary imaging using blood clearing, balloon sinuplasty, sinus stenting, arthroscopy, ophthalmology, ear research, veterinary use and research, etc.

In accordance with at least another aspect of the present disclosure, one or more technique(s) discussed herein may be employed as or along with features to reduce the cost of at least one of manufacture and maintenance of the one or more apparatuses, devices, systems, and storage mediums by reducing or minimizing a number of optical and/or processing components and by virtue of the efficient techniques to cut down cost (e.g., physical labor, mental burden, fiscal cost, time and complexity, etc.) of use/manufacture of such apparatuses, devices, systems, and storage mediums.

The following paragraphs describe certain explanatory embodiments. Other embodiments may include alternatives, equivalents, and modifications. Additionally, the explanatory embodiments may include several novel features, and a particular feature may not be essential to some embodiments of the devices, systems, and methods that are described herein.

According to other aspects of the present disclosure, one or more additional devices, one or more systems, one or more methods, and one or more storage mediums using imaging adjustment or correction and/or other technique(s) are discussed herein. Further features of the present disclosure will in part be understandable and will in part be apparent from the following description and with reference to the attached drawings.

### BRIEF DESCRIPTION OF THE DRAWINGS

For the purposes of illustrating various aspects of the disclosure, wherein like numerals indicate like elements, there are shown in the drawings simplified forms that may be employed, it being understood, however, that the disclosure is not limited by or to the precise arrangements and instrumentalities shown. To assist those of ordinary skill in the relevant art in making and using the subject matter hereof, reference is made to the appended drawings and figures, wherein:

FIG. 1 illustrates at least one embodiment of an imaging, continuum robot, or endoscopic apparatus or system in accordance with one or more aspects of the present disclosure;

FIG. 2 is a schematic diagram showing at least one embodiment of an imaging, steerable catheter, or continuum robot apparatus or system in accordance with one or more aspects of the present disclosure;

FIGS. 3A-3B illustrate at least one embodiment example of a continuum robot and/or medical device that may be used with one or more technique(s), including robotic control and/or atraumatic tip technique(s), in accordance with one or more aspects of the present disclosure;

FIGS. 3C-3D illustrate one or more principles of catheter or continuum robot tip manipulation by actuating one or more bending segments of a continuum robot or steerable catheter 104 of FIGS. 3A-3B in accordance with one or more aspects of the present disclosure;

FIG. 4 is a schematic diagram showing at least one embodiment of an imaging, continuum robot, steerable catheter, or endoscopic apparatus or system in accordance with one or more aspects of the present disclosure;

FIG. 5 is a flowchart of at least one embodiment of a method for planning an operation of at least one embodiment of a continuum robot or steerable catheter apparatus or system in accordance with one or more aspects of the present disclosure;

FIG. 6 includes at least one geometric and/or structural configuration for an atraumatic tip of at least one embodiment of robotic control and/or atraumatic tip technique(s) in accordance with one or more aspects of the present disclosure;

FIG. 7 includes at least one geometric and/or structural configuration for an atraumatic tip of at least one embodiment of robotic control and/or atraumatic tip technique(s) in accordance with one or more aspects of the present disclosure;

FIG. 8 at least another geometric and/or structural configuration for an atraumatic tip of at least one embodiment of robotic control and/or atraumatic tip technique(s) in accordance with one or more aspects of the present disclosure;

FIG. 9 illustrates a flowchart for at least one method embodiment for performing correction, adjustment, and/or smoothing for a catheter or probe of a continuum robot device or system that may be used with one or more tip feature(s) and/or technique(s) in accordance with one or more aspects of the present disclosure;

FIG. 10 shows a schematic diagram of an embodiment of a computer or console that may be used with one or more embodiments of an apparatus or system, or one or more methods, discussed herein in accordance with one or more aspects of the present disclosure;

FIG. 11 shows a schematic diagram of at least an embodiment of a system using a computer or processor, a memory, a database, and input and output devices in accordance with one or more aspects of the present disclosure;

FIG. 12 shows a created architecture of or for a regression model(s) that may be used for autonomous navigation, movement detection, and/or control techniques and/or any other technique discussed herein in accordance with one or more aspects of the present disclosure;

FIG. 13 shows a convolutional neural network architecture that may be used for autonomous navigation, movement detection, and/or control techniques and/or any other technique discussed herein in accordance with one or more aspects of the present disclosure;

FIG. 14 shows a created architecture of or for a regression model(s) that may be used for autonomous navigation, movement detection, and/or control techniques and/or any other technique discussed herein in accordance with one or more aspects of the present disclosure; and

FIG. 15 is a schematic diagram of or for a segmentation model(s) that may be used for catheter connection and/or disconnection detection and/or any other technique discussed herein in accordance with one or more aspects of the present disclosure.

### DETAILED DESCRIPTION OF THE PRESENT DISCLOSURE

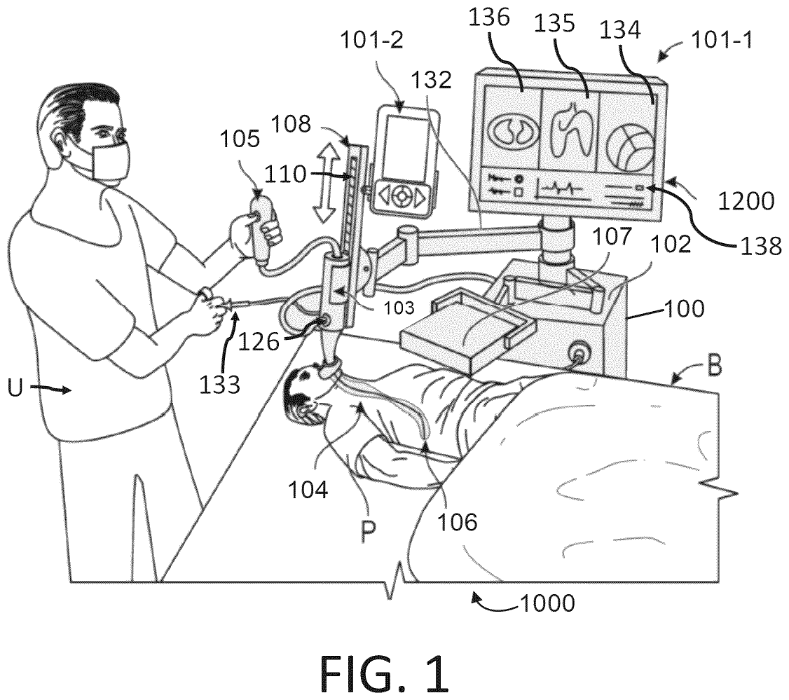

One or more devices, systems, methods and storage mediums for viewing, imaging, and/or characterizing tissue, or an object or sample, for controlling a catheter or probe (e.g., of a steerable imaging device or system, of an endoscope, of a bronchoscope, etc.), and/or for performing or using atraumatic tip feature(s) and/or technique(s) using one or more imaging techniques or modalities (such as, but not limited to, computed tomography (CT), Magnetic Resonance Imaging (MRI), any other techniques or modalities used in imaging (e.g., Optical Coherence Tomography (OCT), Near infrared fluorescence (NIRF), Near infrared auto-fluorescence (NIRAF), Spectrally Encoded Endoscopes (SEE)), etc.) are disclosed herein. Several embodiments of the present disclosure, which may be carried out by the one or more embodiments of an apparatus, system, method, and/or computer-readable storage medium of the present disclosure, are described diagrammatically and visually in FIGS. 1 through 15.

One or more embodiments of the present disclosure avoid the aforementioned issues by providing one or more simple, efficient, cost-effective, and innovative structure that may be used with catheter or probe control technique(s) (including, but not limited to, robotic control technique(s)) as discussed herein and/or atraumatic tip feature(s) and/or technique(s) as discussed herein. In one or more embodiments, the robotic control techniques may be used with a co-registration (e.g., computed tomography (CT) co-registration, cone-beam CT (CBCT) co-registration, etc.) to enhance a successful targeting rate for a predetermined sample, target, or object (e.g., a lung, a portion of a lung, a vessel, a nodule, an organ of a patient, a patient, tissue, etc.) by minimizing human error. CBCT may be used to locate a target, sample, or object (e.g., the lesion(s) or nodule(s) of a lung or airways, plaque or other tissue in one or more samples or in a patient(s), a set or predetermined target in tissue or in a patient, etc.) along with an imaging device (e.g., a steerable catheter, a continuum robot, etc.) and to co-register the target, sample, or object (e.g., the lesions or nodules, plaque or other tissue in one or more samples or in a patient(s), a set or predetermined target in tissue or in a patient, etc.) with the device shown in an image to achieve proper guidance.

Accordingly, it is a broad object of the present disclosure to provide imaging (e.g., computed tomography (CT), Magnetic Resonance Imaging (MRI), etc.) apparatuses, systems, methods, and storage mediums for using a navigation and/or control method or methods (manual or automatic) and/or for using tip or atraumatic tip feature(s) and/or technique(s) in one or more apparatuses or systems (e.g., an imaging apparatus or system, an endoscopic imaging device or system, a bronchoscope, etc.). It is also a broad object of the present disclosure to provide imaging (e.g., computed tomography (CT), Magnetic Resonance Imaging (MRI), etc.) apparatuses, systems, methods, and storage mediums for using a navigation and/or control method or methods for achieving navigation, movement, and/or control through a target, sample, or object (e.g., lung airway(s) during bronchoscopy, a vessel, a patient, an organ, tissue, a portion of a patient, etc.) in one or more apparatuses or systems (e.g., an imaging apparatus or system, an endoscopic imaging device or system, etc.).

Additionally, the navigation and/or control may be employed so that an apparatus or system having multiple portions or sections (e.g., multiple bending portions or sections) operates to: (i) keep track of a path of a portion (e.g., a tip) or of each of the multiple portions or sections of an apparatus or system; (ii) have a state or states of each of the multiple portions or sections match a state or states of a first portion or section of the multiple portions or sections in a case where each portion or section reaches or approaches a same, similar, or approximately similar state (e.g., a position or other state(s) in a target, object, or specimen; a position or other state(s) in a patient; a target position or state(s) in an image or frame; a set or predetermined position or state(s) in an image or frame; a set or predetermined position or state(s) in an image or frame where the first portion or section reaches or approaches the set or predetermined position or state(s) at one point in time and one or more of other portions or sections of the multiple portions or sections reach the set or predetermined position or state(s) at one or more other points in time; any other state (which may include, but is not limited to, an orientation, a position, a pose, a navigation, a path (whether continuous or discontinuous), a state transition, any other desired motion(s) or combination of motion(s) (e.g., one or more features of the present disclosure may assist navigation, orientation, or any other types of motions discussed herein or as desired by a user), etc.), a combination of any state(s) and/or motion(s) discussed herein or desired by a user, etc.) of another portion or section of the one or more devices, systems, methods, and/or storage mediums of the present disclosure, etc.); (iii) utilize additional data (such as, but not limited to, target pose or state information, final pose or state information, interpolated pose or state information, measured pose or state information, converting pose or state information between different states (e.g., drive wire position(s) or state(s); coordinates (three-dimensional (3D) position(s), orientation(s), and/or state(s)); plane and/or angle information for pose(s) or state(s); state position or state information (e.g., target, interpolated, measured pose(s) and/or state(s)); force sensor(s) information; draw or current draw information of one or more actuator motors; section dimension information (e.g., size, shape, length, etc.) for one or more sections of the catheter or probe (e.g., a tip section of the catheter or probe, an atraumatic tip section of the catheter or probe, a middle section of the catheter or probe, a distal section of the catheter or probe (e.g., a section including at least an atraumatic tip piece and/or a capture piece of the catheter or probe), a proximal section of the catheter or probe, any combination thereof, etc.) from an entire device or system (e.g., using forwards or inverse kinematics of the device or system, using other internal sensor(s) or information of the device or system, etc.) and/or external source(s) (e.g., one or more external sensors (e.g., an electromagnetic (EM) sensor, a shape sensor, any other sensor discussed herein or known to those skilled in the art, etc.) in a robotic control algorithm; (iv) utilize differences between a previous or expected robotic control state and a new control state in future calculation(s) of robotic control state(s); (v) address any discontinuous path that may occur (e.g., due to a change (e.g., of a state or states) or other movement or state change/transition of any portion of the apparatus or system), for example, by smoothing out any difference in the discontinuous path over one or more multiple stage positions or states or over one or more other path-like information positions or states, by considering target or object movement(s) (e.g., movement of a patient or a portion of a patient while a probe or catheter is disposed in the patient, etc.); and/or (vi) utilize atraumatic tip feature(s) and/or technique(s), including, but not limited to, providing rapid, accurate, cost-effective, and minimally invasive structure and manufacture/use techniques for structure of a catheter and/or a catheter tip, etc.).

In one or more embodiments, an orientation, pose, or state may include one or more degrees of freedom. For example, in at least one orientation embodiment, two (2) degrees of freedom may be used, which may include an angle for a magnitude of bending and a plane for a direction of bending. In one or more embodiments, matching state(s) may involve matching, duplicating, mimicking, or otherwise copying other characteristics, such as, but not limited to, vectors for each section or portion of the one or more sections or portions of a probe or catheter, for different portions or sections of the catheter or probe. For example, a transition or change from a base angle/plane to a target angle/plane may be set or predetermined using transition values (e.g., while not limited hereto, a base orientation or state may have a stage at 0 mm, an angle at 0 degrees, and a plane at 0 degrees whereas a target orientation or state may have a stage at 20 mm, an angle at 90 degrees, and a plane at 180 degrees. The intermediate values for the stage, angle, and plane may be set depending on how many transition orientations or states may be used).

In one or more embodiments, a continuum robot or steerable catheter may include one or more of the following: (i) a distal bending section or portion, wherein the distal bending section or portion is commanded or instructed automatically or based on an input of a user of the continuum robot or steerable catheter; (ii) a plurality of bending sections or portions including a distal or most distal bending portion or section and the rest of the plurality of the bending sections or portions; and/or (iii) the one or more processors further operate to instruct or command the forward motion, or the motion in the set or predetermined direction, of a motorized linear stage (or other structure used to map path or path-like information) and/or of the continuum robot or steerable catheter automatically and/or based on an input of a user of the continuum robot. A continuum robot or steerable catheter may further include: a base and an actuator that operates to bend the plurality of the bending sections or portions independently; and a motorized linear stage and/or a sensor that operates to move the continuum robot or steerable catheter forward and backward, and/or in the predetermined or set direction or directions, wherein the one or more processors operate to control the actuator and the motorized linear stage and/or the sensor. The plurality of bending sections or portions may each include driving wires that operate to bend a respective section or portion of the plurality of sections or portions, wherein the driving wires are connected to an actuator so that the actuator operates to bend one or more of the plurality of bending sections or portions using the driving wires. One or more embodiments may include a user interface of or disposed on a base, or disposed remotely from a base, the user interface operating to receive an input from a user of the continuum robot or steerable catheter to move one or more of the plurality of bending sections or portions and/or a motorized linear stage and/or a sensor, wherein the one or more processors further operate to receive the input from the user interface, and the one or more processors and/or the user interface operate to use a base coordinate system. One or more displays may be provided to display a path (e.g., a control path) of the continuum robot or steerable catheter. In one or more embodiments, one or more of the following may occur: (i) the continuum robot may further include an operational controller or joystick that operates to issue or input one or more commands or instructions as an input to one or more processors, the input including an instruction or command to move one or more of a plurality of bending sections or portions and/or a motorized linear stage and/or a sensor; (ii) the continuum robot may further include a display to display one or more images taken by the continuum robot; and/or (iii) the continuum robot may further include an operational controller or joystick that operates to issue or input one or more commands or instructions to one or more processors, the input including an instruction or command to move one or more of a plurality of bending sections or portions and/or a motorized linear stage and/or a sensor, and the operational controller or joystick operates to be controlled by a user of the continuum robot. In one or more embodiments, the continuum robot or the steerable catheter may include a plurality of bending sections or portions and may include an endoscope camera, wherein one or more processors operate or further operate to receive one or more endoscopic images from the endoscope camera, and wherein the continuum robot further comprises a display that operates to display the one or more endoscopic images.

Any discussion of a state, pose, position, orientation, navigation, path, or other state type discussed herein is discussed merely as a non-limiting, non-exhaustive embodiment example, and any state or states discussed herein may be used interchangeably/alternatively or additionally with the specifically mentioned type of state. Driving and/or control technique(s) may be employed to adjust, change, or control any state, pose, position, orientation, navigation, path, or other state type that may be used in one or more embodiments for a continuum robot or steerable catheter.

Physicians or other users of the apparatus or system may have reduced or saved labor and/or mental burden using the apparatus or system due to the navigation, control, and/or orientation (or pose, or position, etc.) feature(s) of the present disclosure. Additionally, one or more features of the present disclosure may achieve a minimized or reduced interaction with anatomy (e.g., of a patient), object, or target (e.g., tissue, one or more lungs, one or more airways, etc.) during use, which may reduce the physical and/or mental burden on a patient or target. In one or more embodiments of the present disclosure, a labor of a user to control and/or navigate (e.g., rotate, translate, etc.) the imaging apparatus or system or a portion thereof (e.g., a catheter, a probe, a camera, one or more sections or portions of a catheter, probe, camera, etc.) is saved or reduced via use of the navigation and/or control technique(s) of the present disclosure.

In one or more embodiments, an imaging device or system, or a portion of the imaging device or system (e.g., a catheter, a probe, etc.), the continuum robot, and/or the steerable catheter may include multiple sections or portions, and the multiple sections or portions may be multiple bending sections or portions. In one or more embodiments, the imaging device or system may include manual and/or automatic navigation and/or control features. For example, a user of the imaging device or system (or steerable catheter, continuum robot, etc.) may control each section or portion, and/or the imaging device or system (or steerable catheter, continuum robot, etc.) may operate to automatically control (e.g., robotically control) each section or portion, such as, but not limited to, via one or more navigation, movement, and/or control techniques of the present disclosure.

Navigation, control, and/or orientation feature(s) may include, but are not limited to, implementing mapping of a pose (angle value(s), plane value(s), etc.) of a first portion or section (e.g., a tip portion or section, a distal portion or section, a predetermined or set portion or section, a user selected or defined portion or section, etc.) to a stage position/state (or a position/state of another structure being used to map path or path-like information), controlling angular position(s) of one or more of the multiple portions or sections, controlling rotational orientation or position(s) of one or more of the multiple portions or sections, controlling (manually or automatically (e.g., robotically)) one or more other portions or sections of the imaging device or system (e.g., continuum robot, steerable catheter, etc.) to match or substantially or approximately match (or be close to or similar to) the navigation/orientation/position/pose of the first portion or section in a case where the one or more other portions or sections reach (e.g., subsequently reach, reach at a different time, etc.) the same or similar, or approximately the same or similar, position or state (e.g., in a target, in an object, in a sample, in a patient, in a frame or image, etc.) during navigation in or along a first direction of a path of the imaging device or system, controlling each of the sections or portions of the imaging device or system to retrace and match (or substantially or approximately match or be close/similar to) prior respective position(s) of the sections or portions in a case where the imaging device or system is moving or navigated in a second direction (e.g., in an opposite direction along the path, in a return direction along the path, in a retraction direction along the path, etc.) along the path, etc. For example, an imaging device or system (or portion thereof, such as, but not limited to, a probe, a catheter, a camera, etc.) may enter a target along a path where a first section or portion of the imaging device or system (or portion of the device or system) is used to set the navigation, control, or state path and state(s)/position(s), and each subsequent section or portion of the imaging device or system (or portion of the device or system) is controlled to follow the first section or portion such that each subsequent section or portion matches (or is similar to, approximate to, substantially matching, etc.) the orientation, position, state, etc. of the first section or portion at each location along the path. During retraction, each section or portion of the imaging device or system is controlled to match (or be similar to, be approximate to, be substantially matching, etc.) the prior orientation, position, state, etc. (for each section or portion) for each of the locations along the path. In other words, each section or portion of the device or system may follow a leader (or more than one leader) or may use one or more RFTL and/or FTL technique(s) discussed herein. Additionally or alternatively, as discussed herein, one or more embodiments may use one or more Hold the Line, Close the Gap, Stay the Course, and/or any other control feature(s) of the present disclosure. As such, an imaging or continuum robot device or system (or catheter, probe, camera, etc. of the device or system) may enter and exit a target, an object, a specimen, a patient (e.g., a lung of a patient, an esophagus of a patient, a spline, another portion of a patient, another organ of a patient, a vessel of a patient, etc.), etc. along the same, similar, approximately same or similar, etc. path and using the same orientation, pose, state, etc. for entrance and exit to achieve an optimal navigation, orientation, control, and/or state path. The navigation, control, orientation, and/or state feature(s) are not limited thereto, and one or more devices or systems of the present disclosure may include any other desired navigation, control, orientation, and/or state specifications or details as desired for a given application or use. In one or more embodiments and while not limited thereto, the first portion or section may be a distal or tip portion or section of the imaging or continuum robot device or system. In one or more embodiments, the first portion or section may be any predetermined or set portion or section of the imaging or continuum robot device or system, and the first portion or section may be predetermined or set manually by a user of the imaging or continuum robot device or system or may be set automatically by the imaging device or system (or by a combination of manual and automatic control).

In one or more embodiments of the present disclosure (and while not limited to only this definition), a “change of orientation” or a “change of state” (or a transition of state) may be defined in terms of direction and magnitude. For example, each interpolated step may have a same direction, and each interpolated step may have a larger magnitude as each step approaches a final orientation. Due to kinematics of one or more embodiments, any motion along a single direction may be the accumulation of a small motion in that direction. The small motion may have a unique or predetermined set of wire position or state changes to achieve the orientation change. Large or larger motion(s) in that direction may use a plurality of the small motions to achieve the large or larger motion(s). Dividing a large change into a series of multiple changes of the small or predetermined/set change may be used as one way to perform interpolation. Interpolation may be used in one or more embodiments to produce a desired or target motion, and at least one way to produce the desired or target motion may be to interpolate the change of wire positions or states.

In one or more embodiments of the present disclosure, an apparatus or system may include one or more processors that operate to: instruct or command a distal bending section or portion of a catheter or a probe of the continuum robot such that the distal bending section or portion achieves, or is disposed at, a bending pose or position, the catheter or probe of the continuum robot having a plurality of bending sections or portions and a base; store or obtain the bending pose or position of the distal bending section or portion and store or obtain a position or state of a motorized linear stage (or other structure used to map path or path-like information) that operates to move the catheter or probe of the continuum robot in a case where the one or more processors instruct or command forward motion, or a motion in a set or predetermined direction or directions, of the motorized linear stage (or other predetermined or set structure for mapping path or path-like information); generate a goal or target bending pose or position for each corresponding section or portion of the catheter or probe from, or based on, the previous bending section or portion; generate interpolated poses or positions for each of the sections or portions of the catheter or probe between the respective goal or target bending pose or position and a respective current bending pose or position of each of the sections or portions of the catheter or probe, wherein the interpolated poses or positions are generated such that an orientation vector of the interpolated poses or positions are on a plane that an orientation vector of the respective goal or target bending pose or position and an orientation vector of a respective current bending pose or position create or define; and instruct or command each of the sections or portions of the catheter or probe to move to or be disposed at the respective interpolated poses or positions during the forward motion, or the motion in the set or predetermined direction, of the previous section(s) or portion(s) of the catheter or probe.

In one or more embodiments, the navigation, movement, and/or control may occur such that any intermediate orientations of one or more of the plurality of bending sections or portions is guided towards respective desired, predetermined, or set orientations (e.g., such that the steerable catheter, continuum robot, or other imaging device or system may reach the one or more targets).

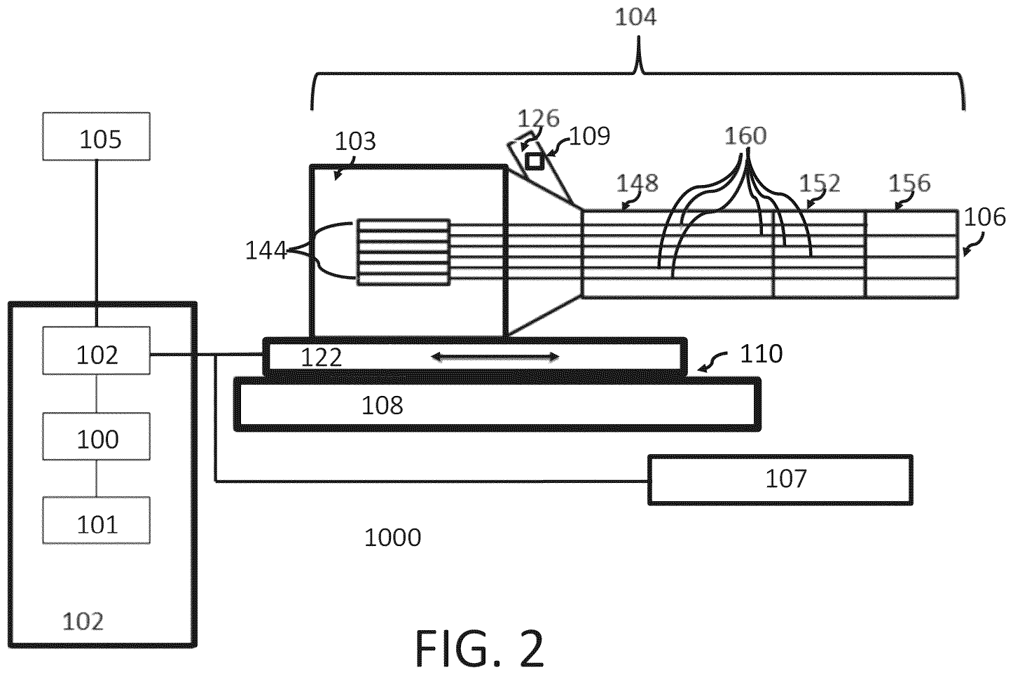

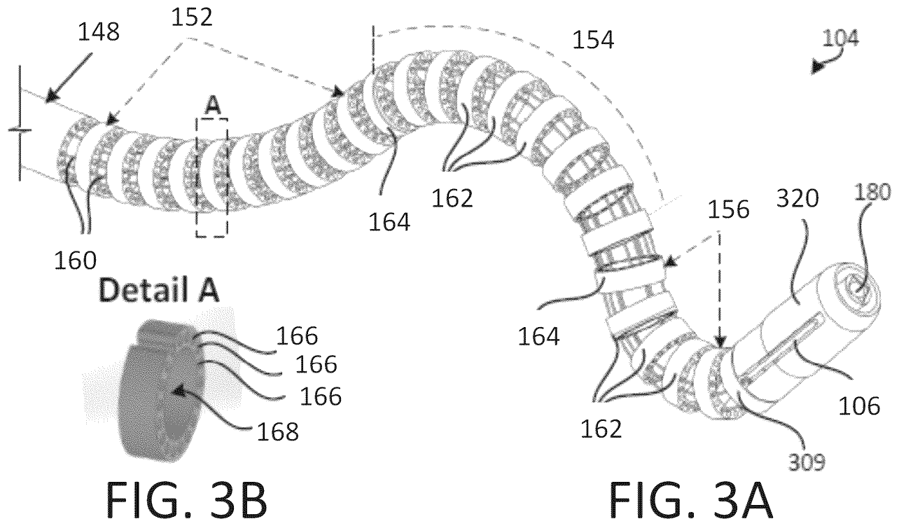

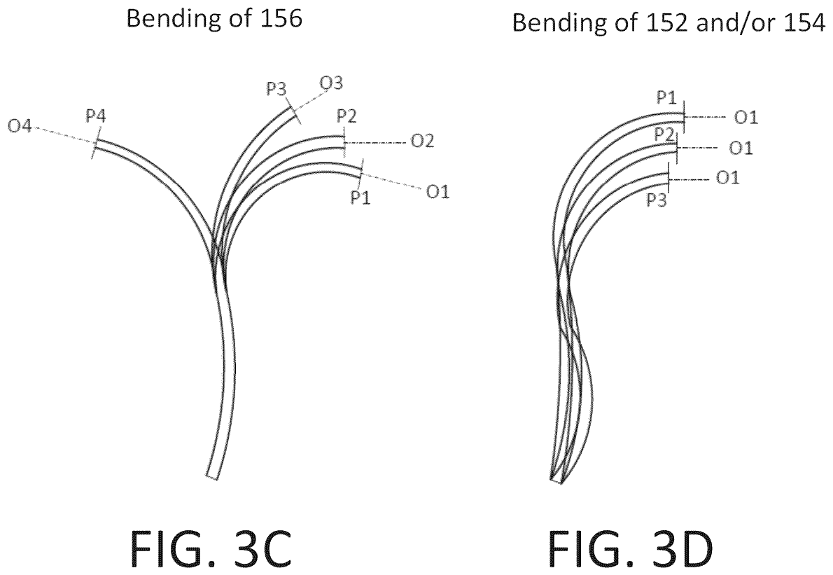

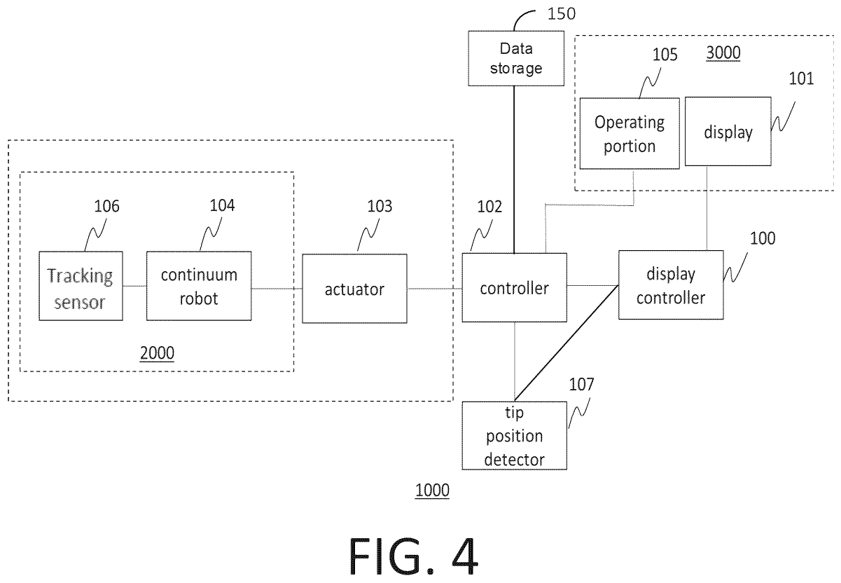

FIG. 1 illustrates a simplified representation of a medical environment, such as an operating room, where a robotic catheter system 1000 may be used. FIG. 2 illustrates a functional block diagram that may be used in at least one embodiment of the robotic catheter system 1000. FIGS. 3A-3D represent at least one embodiment of the catheter 104 (see FIGS. 3A-3B) and bending for the catheter 104 (as shown in FIGS. 3C-3D). FIG. 4 illustrates a logical block diagram that may be used for the robotic catheter system 1000. In at least this embodiment example, the system 1000 may include a computer cart (see e.g., the controller 100, 102 in FIG. 1) operatively connected to a steerable catheter or continuum robot 104 via a robotic platform 108. The robotic platform 108 includes one or more than one robotic arm 132 and a rail 110 (see e.g., FIGS. 1-2) and/or linear translation stage 122 (see e.g., FIG. 2).

As shown in FIGS. 1-4 of the present disclosure, one or more embodiments of a system 1000 for performing robotic control (e.g., for a continuum robot, a steerable catheter, etc.) may include one or more of the following: a display controller 100, a display 101-1, a display 101-2, a controller 102, an actuator 103, a continuum device (also referred to herein as a “steerable catheter” or “an imaging device”) 104, an operating portion 105, a tracking sensor 106 (e.g., an electromagnetic (EM) tracking sensor), a catheter tip position/orientation/pose/state detector 107, and a rail 110 (which may be attached to or combined with a linear translation stage 122) (for example, as shown in at least FIGS. 1-2). The system 1000 may include one or more processors, such as, but not limited to, a display controller 100, a controller 102, a console or computer 1200, a CPU 1201, any other processor or processors discussed herein, etc., that operate to execute a software program, to control the one or more control technique(s), atraumatic tip feature(s)r and/or technique(s), or other feature(s) or technique(s) discussed herein, and to control display of a navigation screen on one or more displays 101-1, 101-2, etc. The one or more processors (e.g., the display controller 100, the controller 102, the console or computer 1200, the CPU 1201, any other processor or processors discussed herein, etc.) may generate a three dimensional (3D) model of a structure (for example, a branching structure like airway of lungs of a patient, an object to be imaged, tissue to be imaged, etc.) based on images, such as, but not limited to, CT images, MRI images, etc. Alternatively, the 3D model may be received by the one or more processors (e.g., the display controller 100, the controller 102, the console or computer 1200, the CPU 1201, any other processor or processors discussed herein, etc.) from another device. A two-dimensional (2D) model may be used instead of 3D model in one or more embodiments. The 2D or 3D model may be generated before a navigation starts. Alternatively, the 2D or 3D model may be generated in real-time (in parallel with the navigation). In the one or more embodiments discussed herein, examples of generating a model of branching structure are explained. However, the models may not be limited to a model of branching structure. For example, a model of a route direct to a target may be used instead of the branching structure. Alternatively, a model of a broad space may be used, and the model may be a model of a place or a space where an observation or a work is performed by using a continuum robot 104 explained below.

In FIG. 1, a user U (e.g., a physician, a technician, etc.) may control the robotic catheter system 1000 via a user interface unit (operation unit) to perform an intraluminal procedure on a patient P positioned on an operating table B. The user interface may include at least one of a main or first display 101-1 (a first user interface unit), a second display 101-2 (a second user interface unit), and a handheld controller 105 (a third user interface unit). The main or first display 101-1 may include, for example, a large display screen attached to the system 1000 and/or the controllers 101, 102 of the system 1000 or mounted on a wall of the operating room and may be, for example, designed as part of the robotic catheter system 1000 or may be part of the operating room equipment. Optionally, there may be a secondary display 101-2 that is a compact (portable) display device configured to be removably attached to the robotic platform 108. Examples of the second or secondary display 101-2 may include, but are not limited to, a portable tablet computer, a mobile communication device (a cellphone), a tablet, a laptop, etc.

The steerable catheter 104 may be actuated via an actuator unit 103. The actuator unit 103 may be removably attached to the robotic platform 108 or any component thereof (e.g., the robotic arm 132, the rail 110, and/or the linear translation stage 122). The handheld controller 105 may include a gamepad-like controller with a joystick having shift levers and/or push buttons, and the controller 105 may be a one-handed controller or a two-handed controller. In one embodiment, the actuator unit 103 may be enclosed in a housing having a shape of a catheter handle. One or more access ports 126 may be provided in or around the catheter handle. The access port 126 may be used for inserting and/or withdrawing end effector tools and/or fluids when performing an interventional procedure of the patient P.

In one or more embodiments, the system 1000 includes at least a system controller 102, a display controller 100, and the main display 101-1. The main display 101-1 may include a conventional display device such as a liquid crystal display (LCD), an OLED display, a QLED display, any other display discussed herein, any other display known to those skilled in the art, etc. The main display 101-1 may provide or display a graphic interface unit (GUI) configured to display one or more views. These views may include a live view image 134, an intraoperative image 135, a preoperative image 136, and other procedural information 138. Other views that may be displayed include a model view, a navigational information view, and/or a composite view. The live image view 134 may be an image from a camera at the tip of the catheter 104. The live image view 134 may also include, for example, information about the perception and navigation of the catheter 104. The preoperative image 136 may include pre-acquired 3D or 2D medical images of the patient P acquired by conventional imaging modalities such as computer tomography (CT), magnetic resonance imaging (MRI), ultrasound imaging, or any other desired imaging modality. The intraoperative image 135 may include images used for image guided procedure such images may be acquired by fluoroscopy or CT imaging modalities (or another desired imaging modality). The intraoperative image 135 may be augmented, combined, or correlated with information obtained from a sensor, camera image, or catheter data.

In the various embodiments where a catheter tip tracking sensor 106 is used, the sensor may be located at the distal end of the catheter 104. The catheter tip tracking sensor 106 may be, for example, an electromagnetic (EM) sensor. If an EM sensor is used, a catheter tip position detector 107 may be included in the robotic catheter system 1000; the catheter tip position detector 107 may include an EM field generator operatively connected to the system controller 102. One or more other embodiments of the catheter/continuum robot 104 may not include or use the EM tracking sensor 106. Suitable electromagnetic sensors for use with a steerable catheter may be used with any feature of the present disclosure, including the sensors discussed, for example, in U.S. Pat. No. 6,201,387 and in International Pat. Pub. WO 2020/194212 A1, which are incorporated by reference herein in their entireties.

While not limited to such a configuration, the display controller 100 may acquire position/orientation/navigation/pose/state (or other state) information of the continuum robot 104 from a controller 102. Alternatively, the display controller 100 may acquire the position/orientation/navigation/pose/state (or other state) information directly from a tip position/orientation/navigation/pose/state (or other state) detector 107. The continuum robot 104 may be a catheter device (e.g., a steerable catheter or probe device). The continuum robot 104 may be attachable/detachable to the actuator 103, and the continuum robot 104 may be disposable.

Similar to FIG. 1, FIG. 2 illustrates the robotic catheter system 1000 including the system controller 102 operatively connected to the display controller 100, which is connected to the first display 101-1 and to the second display 101-2. The system controller 102 is also connected to the actuator 103 via the robotic platform 108 or any component thereof (e.g., the robotic arm 132, the rail 110, and/or the linear translation stage 122). The actuator unit 103 may include a plurality of motors 144 that operate to control a plurality of drive wires 160 (while not limited to any particular number of drive wires 160, FIG. 2 shows that six (6) drive wires 160 are being used in the subject embodiment example). The drive wires 160 travel through the steerable catheter or continuum robot 104. One or more access ports 126 may be located on the catheter 104 (and may include an insertion/extraction detector 109). The catheter 104 may include a proximal section 148 located between the actuator 103 and the proximal bending section 152, where the drive wires 160 operate to actuate the proximal bending section 152. Three of the six drive wires 160 continue through the distal bending section 156 where the drive wires 160 operate to actuate the distal bending section 156 and allow for a range of movement. FIG. 2 is shown with two bendable sections 152, 156 (although one or more bendable sections may be used in one or more embodiments). Other embodiments as described herein may have three bendable sections (see e.g., FIGS. 3A-3D). In some embodiments, a single bending section may be provided, or alternatively, four or more bendable sections may be present in the catheter 104.

FIGS. 3A-3B show at least one embodiment of a continuum robot 104 that may be used in the system 1000 or any other system discussed herein. FIG. 3A shows at least one embodiment of a steerable catheter 104. The steerable catheter 104 may include a non-steerable proximal section 148, a steerable distal section 156, and a catheter tip 320. The proximal section 148 and distal bendable section 156 (including portions 152, 154, and 156 in FIG. 3A) are joined to each other by a plurality of drive wires 160 arranged along the wall of the catheter 104. The proximal section 148 is configured with through-holes (or thru-holes) or grooves or conduits to pass drive wires 160 from the distal section 152, 154, 156 to the actuator unit 103. The distal section 152, 154, 156 is comprised of a plurality of bending segments including at least a distal segment 156, a middle segment 154, and a proximal segment 152. Each bending segment is bent by actuation of at least some of the plurality of drive wires 160 (driving members). The posture of the catheter 104 may be supported by supporting wires (support members) also arranged along the wall of the catheter 104 (as discussed in U.S. Pat. Pub. US2021/0308423, which is incorporated by reference herein in its entirety). The proximal ends of drive wires 160 are connected to individual actuators or motors 144 of the actuator unit 103, while the distal ends of the drive wires 160 are selectively anchored to anchor members in the different bending segments of the distal bendable section(s) 152, 154, 156.

Each bending segment is formed by a plurality of ring-shaped components (rings) with through-holes (or thru-holes), grooves, or conduits along the wall of the rings. The ring-shaped components are defined as wire-guiding members 162 or anchor members 164 depending on a respective function(s) within the catheter 104. The anchor members 164 are ring-shaped components onto which the distal end of one or more drive wires 160 are attached in one or more embodiments. The wire-guiding members 162 are ring-shaped components through which some drive wires 160 slide through (without being attached thereto).

As shown in FIG. 3B, detail “A” obtained from the identified portion of FIG. 3A illustrates at least one embodiment of a ring-shaped component (a wire-guiding member 162 or an anchor member 164). Each ring-shaped component 162, 164 may include a central opening which may form a tool channel 168 and may include a plurality of conduits 166 (grooves, sub-channels, or through-holes (or thru-holes)) arranged lengthwise (and which may be equidistant from the central opening) along the annular wall of each ring-shaped component 162, 164. Inside the ring-shaped component(s) 162, 164, an inner cover, such as is described in U.S. Pat. Pub. US2021/0369085 and US2022/0126060, which are incorporated by reference herein in their entireties, may be included to provide a smooth inner channel and to provide protection. The non-steerable proximal section 148 may be a flexible tubular shaft and may be made of extruded polymer material. The tubular shaft of the proximal section 148 also may have a central opening or tool channel 168 and plural conduits 166 along the wall of the shaft surrounding the tool channel 168. An outer sheath may cover the tubular shaft and the steerable section 152, 154, 156. In this manner, at least one tool channel 168 formed inside the steerable catheter 104 provides passage for an imaging device and/or end effector tools from the insertion port 126 to the distal end of the steerable catheter 104.

The actuator unit 103 may include, in one or more embodiments, one or more servo motors or piezoelectric actuators. The actuator unit 103 may operate to bend one or more of the bending segments of the catheter 104 by applying a pushing and/or pulling force to the drive wires 160.

As shown in FIG. 3A, each of the three bendable segments of the steerable catheter 104 has a plurality of drive wires 160. If each bendable segment is actuated by three drive wires 160, the steerable catheter 104 has nine driving wires arranged along the wall of the catheter 104. Each bendable segment of the catheter 104 is bent by the actuator unit 103 by pushing or pulling at least one of these nine drive wires 160. Force is applied to each individual drive wire in order to manipulate/steer the catheter 104 to a desired pose. The actuator unit 103 assembled with steerable catheter 104 may be mounted on the robotic platform 108 or any component thereof (e.g., the robotic arm 132, the rail 110, and/or the linear translation stage 122). The robotic platform 108, the rail 110, and/or the linear translation stage 122 may include a slider and a linear motor. In other words, the robotic platform 108 or any component thereof (e.g., the robotic arm 132, the rail 110, and/or the linear translation stage 122) is motorized, and may be controlled by the system controller 102 to insert and remove the steerable catheter 104 to/from the target, sample, or object (e.g., the patient, the patient's bodily lumen, one or more airways, a lung, a tissue, an organ, a target or object, a specimen, etc.).

An imaging device 180 that may be inserted through the tool channel 168 includes an endoscope camera (videoscope) along with illumination optics (e.g., optical fibers or LEDs) (or any other camera or imaging device, tool, etc. discussed herein or known to those skilled in the art). The illumination optics provide light to irradiate the lumen and/or a lesion target which is a region of interest within the target, sample, or object (e.g., in a patient). End effector tools may refer to endoscopic surgical tools including clamps, graspers, scissors, staplers, ablation or biopsy needles, and other similar tools, which serve to manipulate body parts (organs or tumorous tissue) during imaging, examination, or surgery. The imaging device 180 may be what is commonly known as a chip-on-tip camera and may be color (e.g., take one or more color images) or black-and-white (e.g., take one or more black-and-white images). In one or more embodiments, a camera may support color and black-and-white images. In one or more embodiments, the imaging device 180 or camera may use fluoroscopy (NIRF, NIRAF, any other fluoroscopy discussed herein or known to those skilled in the art, etc.) in addition to another imaging modality (e.g., OCT, IVUS, any other imaging modality discussed herein or known to those skilled in the art, etc.).

In some embodiments, a tracking sensor 106 (e.g., an EM tracking sensor) is attached to or installed in the catheter tip 320. In this embodiment, the steerable catheter 104 and the tracking sensor 106 may be tracked by the tip position detector 107. Specifically, the tip position detector 107 detects a position of the tracking sensor 106, and outputs the detected positional information to the system controller 102. The system controller 102, receives the positional information from the tip position detector 107, and continuously records and displays the position of the steerable catheter 104 with respect to the coordinate system of the target, sample, or object (e.g., a patient, a lung, an airway(s), a vessel, etc.). The system controller 102 operates to control the actuator unit 103 and the robotic platform 108 or any component thereof (e.g., the robotic arm 132, the rail 110, and/or the linear translation stage 122) in accordance with the manipulation commands input by the user U via one or more of the input and/or display devices (e.g., the handheld controller 105, a GUI at the main display 101-1, touchscreen buttons at the secondary display 101-2, etc.).

FIG. 3C and FIG. 3D show exemplary catheter tip manipulations by actuating one or more bending segments of the steerable catheter 104. As illustrated in FIG. 3C, manipulating only the most distal segment 156 of the steerable section may change the position and orientation of the catheter tip 320. On the other hand, manipulating one or more bending segments (152 or 154) other than the most distal segment may affect only the position of catheter tip 320, but may not affect the orientation of the catheter tip 320. In FIG. 3C, actuation of distal segment 156 changes the catheter tip from a position P1 having orientation O1, to a position P2 having orientation O2, to position P3 having orientation O3, to position P4 having orientation O4, etc. In FIG. 3D, actuation of the middle segment 152 and/or the middle segment 154 may change the position of the catheter tip 320 from a position P1 having orientation O1 to a position P2 and position P3 having the same orientation O1. Here, it should be appreciated by those skilled in the art that exemplary catheter tip manipulations shown in FIG. 3C and FIG. 3D may be performed during catheter navigation (e.g., while inserting the catheter 104 through tortuous anatomies, one or more targets, one or more lungs or other organs, one or more airways, samples, objects, a patient, etc.). In the present disclosure, the one or more catheter tip manipulations shown in FIG. 3C and FIG. 3D may apply namely to the targeting mode applied after the catheter tip 320 has been navigated to a predetermined distance (a targeting distance) from the target, sample, or object.

The actuator 103 may proceed or retreat along a rail 110 (e.g., to translate the actuator 103, the continuum robot/catheter 104, etc.), and the actuator 103 and continuum robot 104 may proceed or retreat in and out of the patient's body or other target, object, or specimen (e.g., tissue). As shown in FIG. 3B, the catheter device 104 may include a plurality of driving backbones and may include a plurality of passive sliding backbones. In one or more embodiments, the catheter device 104 may include at least nine (9) driving backbones and at least six (6) passive sliding backbones. The catheter device 104 may include an atraumatic tip at the end of the distal section of the catheter device 104.

FIG. 4 illustrates that a system 1000 may include the system controller 102 which may operate to execute software programs and control the display controller 100 to display a navigation screen (e.g., a live view image 134) on the main display 101-1 and/or the secondary display 101-2. The display controller 100 may include a graphics processing unit (GPU) or a video display controller (VDC) (or any other suitable hardware discussed herein or known to those skilled in the art.

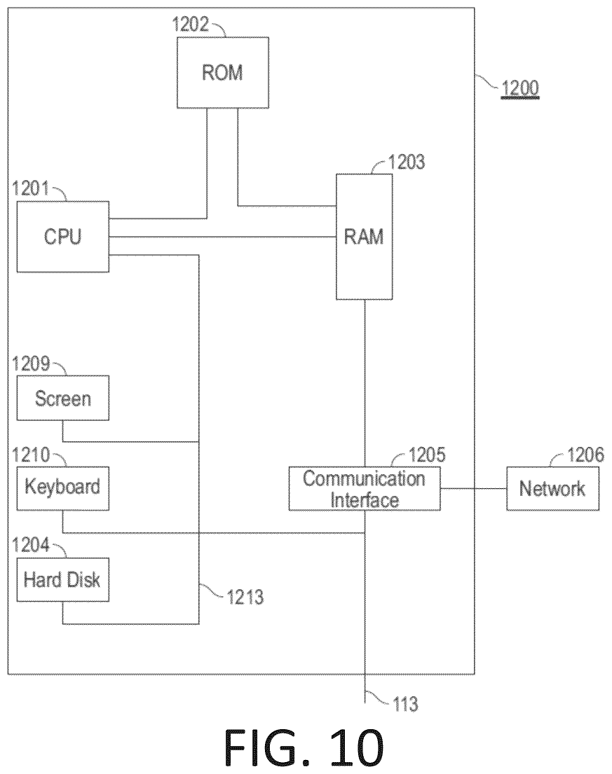

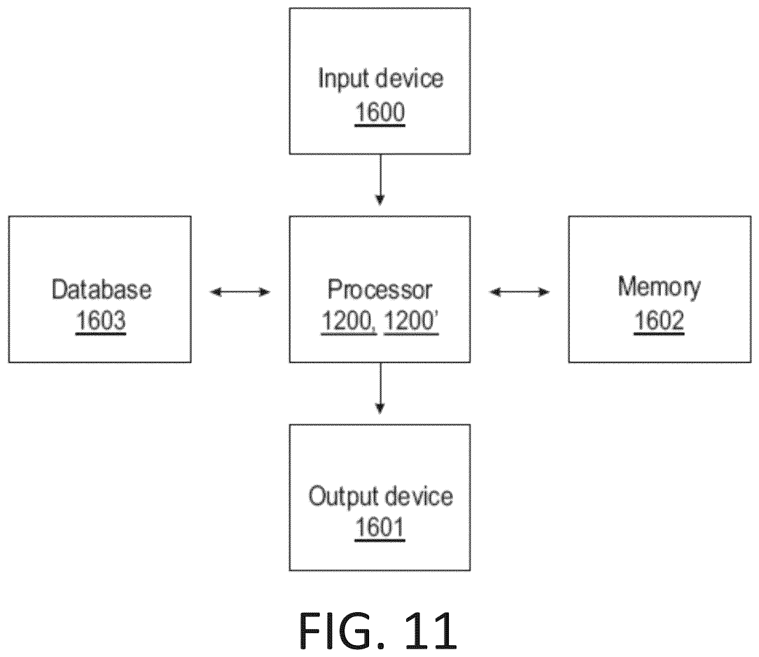

In one or more embodiments, the system controller 102 and/or the display controller 100 may include one or more computer or processing components or units, such as, but not limited to, the components, processors, or units shown in at least FIG. 10 discussed further below. The system controller 102 and the display controller 100 may be configured separately. Alternatively, the system controller 102 and the display controller 100 may be configured as one device. In either case, the system controller 102 and the display controller 100 may include substantially the same components in one or more embodiments. For example, as shown in FIG. 10, the system controller 102 and the display controller 100 may include a central processing unit (CPU 1201) (which may be comprised of one or more processors (microprocessors)), a random access memory (RAM 1203) module, an input/output or communication (I/O 1205) interface, a read only memory (ROM 1202), and data storage memory (e.g., a hard disk drive 1204 or solid state drive (SSD) 1204) (see e.g., also data storage 150 of FIG. 4). In the embodiments described below, the navigation screen is a graphical user interface (GUI) generated by a software program but, it may also be generated by firmware, or a combination of software and firmware. A Solid State Drive (SSD) 1204 may be used instead of HDD 1204 as the data storage 150. In one or more additional embodiments, the one or more processors, and/or the display controller 100 and/or the controller 102, may include structure as shown in FIG. 10 and/or in FIGS. 11-15 as further discussed below.

The system controller 102 may control the steerable catheter 104 based on any known kinematic algorithms applicable to continuum or steerable catheter robots. For example, the segments or portions of the steerable catheter 104 may be controlled individually to direct the catheter tip with a combined actuation of all bendable segments or sections. By way of another example, a controller 102 may control the catheter 104 based on an algorithm known as follow the leader (FTL) algorithm. By applying FTL, the most distal segment 156 is actively controlled with forward kinematic values, while the middle segment 154 and the other middle or proximal segment 152 (following sections) of the steerable catheter 104 move at a first position in the same way as the distal section moved at the first position or a second position near the first position (e.g., the subsequent sections may follow a path traced out by the distal section). In one or more embodiments, the RFTL algorithm may be used. For example, in one or more embodiments, to withdraw the catheter 104, a reverse IFTL (RFTL) process may be implemented. This may be implemented using inverse kinematics. The RFTL mode may automatically control all sections of the steerable catheter 104 to retrace the pose (or state) from the same position along the path made during insertion (e.g., in a reverse or backwards order or manner).

The display controller 100 may acquire position information of the steerable catheter 104 from system controller 102. Alternatively, the display controller 100 may acquire the position information directly from the tip position detector 107. The steerable catheter 104 may be a single-use or limited-use catheter device. In other words, the steerable catheter 104 may be attachable to, and detachable from, the actuator unit 103 to be disposable.

The tool may be a medical tool such as an endoscope camera, forceps, a needle, or other biopsy or ablation tools. In one embodiment, the tool may be described as an operation tool or working tool. The working tool is inserted or removed through the working tool access port 126. In the embodiments below, at least one embodiment of using a steerable catheter 104 to guide a tool to a target is explained. The tool may include an endoscope camera or an end effector tool, which may be guided through a steerable catheter under the same principles. In a procedure there is usually a planning procedure, a registration procedure, a targeting procedure, and an operation procedure.

The one or more processors, such as, but not limited to, the display controller 100, may generate and output a navigation screen to the one or more displays 101-1, 101-2 based on the 2D/3D model and the position/orientation/navigation/pose/state (or other state) information by executing the software. The navigation screen may indicate a current position/orientation/navigation/pose/state (or other state) of the continuum robot 104 on the 2D/3D model. By using the navigation screen, a user may recognize the current position/orientation/navigation/pose/state (or other state) of the continuum robot 104 in the branching structure. Any feature of the present disclosure may be used with any navigation/pose/state feature(s) or other feature(s) discussed in U.S. Prov. Pat. App. No. 63/504,972, filed on May 30, 2023, the disclosure of which is incorporated by reference herein in its entirety, and International App. No. PCT/US2024/031766, filed May 30, 2024, the disclosure of which is incorporated by reference herein in its entirety. By observing the navigation screen, a user may recognize the current position of the steerable catheter 104 in the branching structure. Upon completing navigation to a desired target, one or more end effector tools may be inserted through the access port 126 at the proximal end of the catheter 104, and such tools may be guided through the tool channel 168 of the catheter bodyto perform an intraluminal procedure from the distal end of the catheter 104.

The ROM 1202 and/or HDD 1204 may operate to store the software in one or more embodiments. The RAM 1203 may be used as a work memory. The CPU 1201 may execute the software program developed in the RAM 1203. The I/O or communication interface 1205 may operate to input the positional (or other state) information to the display controller 100 (and/or any other processor discussed herein) and to output information for displaying the navigation screen to the one or more displays 101-1, 101-2. In the embodiments below, the navigation screen may be generated by the software program. In one or more other embodiments, the navigation screen may be generated by a firmware.

One or more devices or systems, such as the system 1000, may include a tip position/orientation/navigation/pose/state (or other state) detector 107 that operates to detect a position/orientation/navigation/pose/state (or other state) of the EM tracking sensor 106 and to output the detected positional (and/or other state) information to the controller 100 or 102 (e.g., as shown in FIGS. 1-2), or to any other processor(s) discussed herein.

The controller 102 may operate to receive the positional (or other state) information of the tip of the continuum robot 104 from the tip position/orientation/navigation/pose/state (or any other state discussed herein) detector 107. The controller 100 and/or the controller 102 operates to control the actuator 103 in accordance with the manipulation by a user (e.g., manually), and/or automatically (e.g., by a method or methods run by one or more processors using software, by the one or more processors, using automatic manipulation in combination with one or more manual manipulations or adjustments, etc.) via one or more operation/operating portions or operational controllers 105 (e.g., such as, but not limited to a joystick as shown in FIGS. 1-2; see also, diagram of FIG. 4). The one or more displays 101-1, 101-2 and/or operation portion or operational controllers 105 may be used as a user interface 3000 (also referred to as a receiving device) (e.g., as shown diagrammatically in FIG. 4). In an embodiment shown in FIGS. 1-2 or the embodiment shown in FIG. 4, the system(s) 1000 may include, as an operation unit, the display 101-1 (e.g., such as, but not limited to, a large screen user interface with a touch panel, first user interface unit, etc.), the display 101-2 (e.g., such as, but not limited to, a compact user interface with a touch panel, a second user interface unit, etc.) and the operating portion 105 (e.g., such as, but not limited to, a joystick shaped user interface unit having shift lever/button, a third user interface unit, a gamepad, or other input device, etc.).

The controller 100 and/or the controller 102 (and/or any other processor discussed herein) may control the continuum robot 104 based on an algorithm known as follow the leader (FTL) algorithm and/or the RFTL algorithm. The FTL algorithm may be used in addition to the robotic control features of the present disclosure. For example, by applying the FTL algorithm, the middle section and the proximal section (following sections) of the continuum robot 104 may move at a first position (or other state) in the same or similar way as the distal section moved at the first position (or other state) or a second position (or state) near the first position (or state) (e.g., during insertion of the continuum robot/catheter 104, by using the navigation, movement, and/or control feature(s) of the present disclosure, etc.). Similarly, the middle section and the distal section of the continuum robot 104 may move at a first position or state in the same/similar/approximately similar way as the proximal section moved at the first position or state or a second position or state near the first position (e.g., during removal of the continuum robot/catheter 104). Additionally or alternatively, the continuum robot/catheter 104 may be removed by automatically and/or manually moving along the same or similar, or approximately same or similar, path that the continuum robot/catheter 104 used to enter a target (e.g., a body of a patient, an object, a specimen (e.g., tissue), etc.) using the FTL algorithm, including, but not limited to, using FTL with the one or more control, atraumatic tip, or other technique(s) discussed herein.

Additionally or alternatively, any feature of the present disclosure may be used with features, including, but not limited to, training feature(s), autonomous navigation feature(s), artificial intelligence feature(s), etc., as discussed in U.S. Prov. Pat. App. No. 63/513,803, filed on Jul. 14, 2023, the disclosure of which is incorporated by reference herein in its entirety, U.S. Prov. Pat. App. 63/513,794, filed Jul. 14, 2023, the disclosure of which is incorporated by reference herein in its entirety, U.S. Prov. Pat. App. No. 63/587,637, filed Oct. 3, 2023, the disclosure of which is incorporated by reference herein in its entirety, U.S. Prov. Pat. App. No. 63/603,523, filed Nov. 28, 2023, the disclosure of which is incorporated by reference herein in its entirety, International App. No. PCT/US2024/037930, filed Jul. 12, 2024, the disclosure of which is incorporated by reference herein in its entirety, International App. No. PCT/US2024/037935, filed Jul. 12, 2024, the disclosure of which is incorporated by reference herein in its entirety, and International App. No. PCT/US2024/037924, filed Jul. 12, 2024, the disclosure of which is incorporated by reference herein in its entirety.

Any of the one or more processors, such as, but not limited to, the controller 102 and the display controller 100, may be configured as one device (for example, the structural attributes of the controller 100 and the controller 102 may be combined into one controller or processor, such as, but not limited to, the one or more other processors discussed herein (e.g., computer, console, or processor 1200, etc.).

The system 1000 may include a tool channel 126 for a camera, biopsy tools, or other types of medical tools (as shown in FIGS. 1-2). For example, the tool may be a medical tool, such as an endoscope, a forceps, a needle, or other biopsy tools, etc. In one or more embodiments, the tool may be described as an operation tool or working tool (e.g., an imaging device, a camera, etc.). The working tool may be inserted or removed through a working tool insertion slot 126 (as shown in FIGS. 1-2). Any of the features of the present disclosure may be used in combination with any of the features, including, but not limited to, the tool insertion slot, as discussed in U.S. Prov. Pat. App. No. 63/378,017, filed Sep. 30, 2022, the disclosure of which is incorporated by reference herein in its entirety, as discussed in U.S. patent application Ser. No. 18/477,081, filed Sep. 28, 2023, the disclosure of which is incorporated by reference herein in its entirety, and/or any of the features as discussed in U.S. Prov. Pat. App. No. 63/377,983, filed Sep. 30, 2022, the disclosure of which is incorporated by reference herein in its entirety, and as discussed in U.S. Prov. Pat. App. No. 63/383,210, filed Nov. 10, 2022, the disclosure of which is incorporated by reference herein in its entirety.

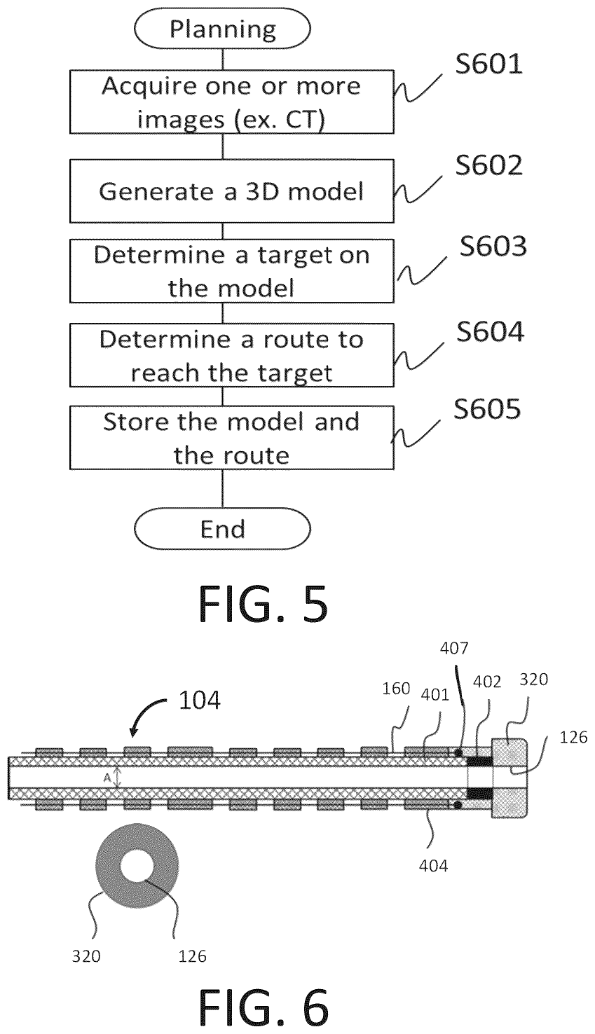

One or more of the features discussed herein may be used for planning procedures, including using one or more models for robotic control and/or artificial intelligence applications. As an example of one or more embodiments, FIG. 5 is a flowchart showing steps of at least one planning procedure of an operation of the continuum robot/catheter device 104. One or more of the processors discussed herein may execute the steps shown in FIG. 5, and these steps may be performed by executing a software program read from a storage medium, including, but not limited to, the ROM 1202 or HDD/SSD 1204, by CPU 1201 or by any other processor discussed herein. One or more methods of planning using the continuum robot/catheter device 104 may include one or more of the following steps: (i) In step s601, one or more images such, as CT or MRI images, may be acquired; (ii) In step s602, a three dimensional model of a branching structure (for example, an airway model of lungs or a model of an object, specimen or other portion of a body) may be generated based on the acquired one or more images; (iii) In step s603, a target on the branching structure may be determined (e.g., based on a user instruction, based on preset or stored information, etc.); (iv) In step s604, a route of the continuum robot/catheter device 104 to reach the target (e.g., on the branching structure) may be determined (e.g., based on a user instruction, based on preset or stored information, based on a combination of user instruction and stored or preset information, etc.); and/or (v) In step s605, the generated model (e.g., the generated two-dimensional or three-dimensional model) and the decided route on the model may be stored (e.g., in the RAM 1203 or HDD/SSD or data storage 1204/150, in any other storage medium discussed herein, in any other storage medium known to those skilled in the art, etc.). In this way, a model (e.g., a 2D or 3D model) of a branching structure may be generated, and a target and a route on the model may be determined and stored before the operation of the continuum robot 104 is started.

In one or more of the embodiments below, embodiments of using a catheter device/continuum robot 104 are explained, such as, but not limited to features for performing navigation, movement, and/or robotic control technique(s), performing or using atraumatic tip feature(s) and/or technique(s), or any other feature(s) and/or technique(s) discussed herein.

Pose or state information may be stored in a lookup table or tables, and the pose or state information for one or more sections of the catheter or probe may be updated in the lookup table based on new information (e.g., environmental change(s) for the catheter or probe, movement of a target or sample, movement of a patient, user control, relaxation state changes, etc.). The new information or the updated information may be used to control the one or more sections of the catheter or probe more efficiently during navigation (forwards and/or backwards). For example, in a case where a previously stored pose or state may have shifted or changed due to a movement or relaxation of the target, object, or sample (e.g., a patient may move), the previously stored pose or state may not be ideal or may work less efficiently as compared with an updated pose or state modified or updated in view of the new information (e.g., the movement, in this example). As such, one or more embodiments of the present disclosure may update or modify the pose or state information such that robotic control of the catheter or probe may work efficiently in view of the new information, movement, relaxation, and/or environmental change(s). In addition to having the update or change affect the previously stored history or known history at that point in space (e.g., similar to dragging that point (e.g., of the target, object, or sample (e.g., a patient, a portion of a patient, a vessel, a spline, a lung, etc.); of the catheter or probe; etc.) and recalculating the path), in one or more embodiments, the update or change may also affect a number of other points (e.g., all points in a lookup table or tables, all points forward beyond the initially changed point, one or more future points or points beyond the initially changed point as desired, etc.). For example, in one or more embodiments, the transform (or difference, change, update, etc.) between the previous pose or state and the new or updated post or state may be propagated to all points going forward or may be propagated to one or more of forward points (e.g., for a predetermined or set range, for a predetermined or set distance, etc.). Doing so in one or more embodiments may operate to shift all or part of the future path based on how the pose or state of the catheter or probe was adjusted, using that location as a pivot point. Such update(s) may be obtained from one or more internal sources (e.g., one or more processors, one or more sensors, combination(s) thereof, etc.) or may be obtained from one or more external sources (e.g., one or more other processors, one or more external sensors, combination(s) thereof, etc.). For example, a difference between a real-time target, sample, or object (e.g., an airway) and the previous target, sample, or object (e.g., a previous airway) may be detected using machine vision (of the endoscope image) or using multiple medical images. Body, target, object, or sample divergence may also be estimated from other sensors, like one measuring breathing or the motion of the body (or another predetermined or set motion or change to track). In one or more embodiments, an amount of transform, update, and/or change may be different for each point, and/or may be a function of, for example, a distance from a current point.

One or more robotic control methods of the present disclosure may be employed in one or more embodiments. For example, one or more of the following techniques or methods may be used to update historical information of a catheter or probe (or portion(s) or section(s) of the catheter or probe): Hold the Line, Close the Gap, and/or Stay the Course.

One or more methods of controlling or using a continuum robot/catheter device (e.g., robot or catheter device 104) may use one or more Hold the Line techniques, Close the Gap techniques, and/or Stay the Course techniques, such as, but not limited to, the techniques discussed in U.S. Pat. App. No. 63/585,128 filed on Sep. 25, 2023, the disclosure of which is incorporated herein by reference in its entirety, and as discussed in International Pat. App. No. PCT/US2024/048210, filed Sep. 24, 2024, the disclosure of which is incorporated herein by reference in its entirety. For example, while not limited thereto, at least one Hold the Line method may include one or more of the following steps: (i) In step s700, a catheter or robot device may move forward (e.g., while a stage of the catheter or robot moves forward, while the navigation is mapped to Z stage position (e.g., a position, pose, or state of a Tip section or portion of the catheter or probe may be converted to a coordinate (e.g., X, Y, Z coordinate) during navigation), etc.); (ii) In step s701, coordinates for a Tip end effector of the Tip section or portion may be calculated; (iii) In step S702, add the calculated coordinate information to a 3D path for the Tip end/section/portion and/or catheter or probe; (iv) In step S703, coordinates for a Middle/proximal end effector of a Middle/proximal (or other section or portion subsequent to or following the Tip section or portion) section or portion of the catheter or probe may be calculated; (v) In step S704, a distance from a closest point along the 3D path may be identified for the Middle/proximal end effector and/or the Tip end effector; (vi) In step S706, the calculated distance may be converted to a change in a pose, position, or state of the Tip end effector, the Middle/proximal end effector, and/or the catheter or probe; and (vii) In step S707, the pose, position, or state of the Middle/proximal section or portion of the catheter or probe may be updated (e.g., to match the pose, position or state of the Tip section or portion of the catheter or probe at that point along the path) and the process may then return to step S703 (and repeat steps S703 through S704 and/or S705 as needed). As another example, while not limited thereto, one or more Close the Gap methods may include one or more of the following: (i) In step s800, a pose, position, or state of a Middle/proximal section or portion of a catheter or probe may be identified, determined, calculated, or otherwise obtained; (ii) In step s801, a pose, position, or state of a Tip section or portion of a catheter or probe may be identified, determined, calculated, or otherwise obtained; (iii) In step S802, a difference between the poses, positions, or states of the Tip section or portion and of the Middle/proximal (or other subsequent or following) section or portion may be determined, identified, calculated, or otherwise obtained; (iv) In step S804, the pose, position, or state difference between the tip section or portion and the Middle/proximal (or other subsequent or following) section or portion may be interpolated over a set or predetermined length; and (v) In step S805, the pose, position, or state of the Middle/proximal (or other subsequent or following) section or portion of the catheter or probe may be updated using the corresponding interpolated pose, position, or state difference. By way of another example, while not limited thereto, one or more Stay the Course methods may include one or more of the following: (i) In step s900, a catheter or robot device may move forward (e.g., while a stage of the catheter or robot moves forward, while the navigation is mapped to Z stage position (e.g., a position, pose, or state of a Tip section or portion of the catheter or probe may be converted to a coordinate (e.g., X, Y, Z coordinate) during navigation), etc.; in one or more embodiments, step S900 may be performed similarly or substantially similar to, or the same as, step S700 described above); (ii) In step s901, a vector (e.g., a normal vector or a normal path; a predetermined, targeted, desired trajectory or path; etc.) may be calculated for a Tip end effector of the Tip section or portion of the catheter or probe; (iii) In step S903, a deviation of the Tip end effector from the normal path or vector due to catheter or probe shape and/or motion (e.g., motion from movement of a stage or translational stage, motion from changes due to an environment or the target or sample in which the catheter or probe is located, body divergence, motion from another source, etc.); (iv) In step S904, a change to a pose, position, or state of a Middle/proximal (or other section or portion subsequent to or following the Tip section or portion) section or portion of the catheter or probe may be calculated to counteract or remove the calculated deviation (e.g., from step S903); and (v) In step S905, the pose, position, or state of the Middle/proximal section or portion of the catheter or probe may be updated (e.g., to match the pose, position or state of the Tip section or portion of the catheter or probe at that point along the path, to eliminate or remove the calculated deviation, etc.), and/or a proximal section(s) and/or the stage may be updated or adjusted. In one or more embodiments, one or more methods may include a step S902 in which it is evaluated or determined whether a path deviation due to a catheter or probe shape and/or motion (e.g., due to stage motion, due to translational motion, due to movement or motion of the target, object, or sample, body divergence, due to motion of an outside force or influence on the catheter or probe, etc.) exists. If “YES”, then the process may proceed to steps S903-S905 (and may repeat steps S903-S905 as needed). If “NO”, then the process may end. In one or more embodiments, the existence of the path deviation of step S902 may be used as a trigger for, and used in, the calculation of the step S903.

As aforementioned, a catheter or probe may be controlled to stay the desired course. For example, a pose, position, or state of a section or sections, or of a portion or portions, of the catheter or probe may be adjusted to minimize any deviation of a pose, position, or state of one or more next (e.g., subsequent, following, proximal, future, Middle/proximal, etc.) sections out of the predetermined, targeted, desired trajectory and maximum motion along the trajectory. In one or more embodiments, the coordinates and the trajectory of subsequent/following/next/future sections may be known, set, or determined, and information for one or more prior sections may be known, set, or determined. By considering section lengths in one or more embodiments, one or more advantageous results may be achieved. By using one or more features of the present disclosure, any counter-active or undesired motion(s) may be avoided or eliminated.

In one or more embodiments, the system controller 102 (or any other controller, processor, computer, etc. discussed herein) may operate to perform a robotic control mode and/or an autonomous navigation mode. During the robotic control mode and/or the autonomous navigation mode, the user does not need to control the bending and translational insertion position of the steerable catheter 104. The autonomous navigation mode may include or comprise: (1) a perception step, (2) a planning step, and (3) a control step. In the perception step, the system controller 102 may receive an endoscope view (or imaging data) and may analyze the endoscope view (or imaging data) to find addressable airways from the current position/orientation of the steerable catheter 104. At an end of this analysis, the system controller 102 identifies or perceives these addressable airways as paths in the endoscope view (or imaging data).

The planning step is a step to determine a target path, which is the destination for the steerable catheter 104. While there are a couple of different approaches to select one of the paths as the target path, the present disclosure uniquely includes means to reflect user instructions concurrently for the decision of a target path among the identified or perceived paths. Once the system 1000 determines the target paths while considering concurrent user instructions, the target path is sent to the next step, i.e., the control step.

The control step is a step to control the steerable catheter 104 and the linear translation stage 122 (or any other portion of the robotic platform 108) to navigate the steerable catheter 104 to the target path, pose, state, etc. This step may also be performed as an automatic step. The system controller 102 operates to use information relating to the real time endoscope view (e.g., the view 134), the target path, and an internal design & status information on the robotic catheter system 1000.

As shown in FIG. 1, the real-time endoscope view 134 may be displayed in a main display 101-1 (as a user input/output device) in the system 1000. The user may see the airways in the real-time endoscope view 134 through the main display 101-1. This real-time endoscope view 134 may also be sent to the system controller 102. In the perception step, the system controller 102 may process the real-time endoscope view 134 and may identify path candidates by using image processing algorithms. Among these path candidates, the system controller 102 may select the paths with the designed computation processes, and then may display the paths with a circle, octagon, or other geometric shape with the real-time endoscope view 134, for example, as discussed in: U.S. Prov. Pat. App. No. 63/513,803, filed on Jul. 14, 2023, the disclosure of which is incorporated by reference herein in its entirety, U.S. Prov. Pat. App. 63/513,794, filed Jul. 14, 2023, the disclosure of which is incorporated by reference herein in its entirety, U.S. Prov. Pat. App. No. 63/587,637, filed Oct. 3, 2023, the disclosure of which is incorporated by reference herein in its entirety, U.S. Prov. Pat. App. No. 63/603,523, filed Nov. 28, 2023, the disclosure of which is incorporated by reference herein in its entirety, International App. No. PCT/US2024/037930, filed Jul. 12, 2024, the disclosure of which is incorporated by reference herein in its entirety, International App. No. PCT/US2024/037935, filed Jul. 12, 2024, the disclosure of which is incorporated by reference herein in its entirety, and International App. No. PCT/US2024/037924, filed Jul. 12, 2024, the disclosure of which is incorporated by reference herein in its entirety.

In planning step, the system controller 102 may provide a cursor so that the user may indicate the target path by moving the cursor with the joystick 105. When the cursor is disposed or is located within the area of the path, the system controller 102 operates to recognize the path with the cursor as the target path.

In a further embodiment example, the system controller 102 may pause the motion of the actuator unit 103 and the linear translation stage 122 while the user is moving the cursor so that the user may select the target path with a minimal change of the real-time endoscope view 134 and paths since the system 1000 would not move in such a scenario.

In one or more of the embodiments below, embodiments of using a catheter device/continuum robot 104 are explained. Any feature of the present disclosure may be used with autonomous navigation, movement detection, and/or control technique(s), including, but not limited to, the features discussed in: U.S. Prov. Pat. App. No. 63/513,803, filed on Jul. 14, 2023, the disclosure of which is incorporated by reference herein in its entirety, U.S. Prov. Pat. App. 63/513,794, filed Jul. 14, 2023, the disclosure of which is incorporated by reference herein in its entirety, U.S. Prov. Pat. App. No. 63/587,637, filed Oct. 3, 2023, the disclosure of which is incorporated by reference herein in its entirety, U.S. Prov. Pat. App. No. 63/603,523, filed Nov. 28, 2023, the disclosure of which is incorporated by reference herein in its entirety, International App. No. PCT/US2024/037930, filed Jul. 12, 2024, the disclosure of which is incorporated by reference herein in its entirety, International App. No. PCT/US2024/037935, filed Jul. 12, 2024, the disclosure of which is incorporated by reference herein in its entirety, and International App. No. PCT/US2024/037924, filed Jul. 12, 2024, the disclosure of which is incorporated by reference herein in its entirety.

The system controller 102 may control the steerable catheter 104 based on any known kinematic algorithms applicable to continuum or snake-like catheter robots. For example, the system controller controls the steerable catheter 104 based on an algorithm known as follow the leader (FTL) algorithm or on the RFTL algorithm. By applying the FTL algorithm, the most distal segment 156 is actively controlled with forward kinematic values, while the middle segment 154 and the other middle or proximal segment 152 (following sections) of the steerable catheter 104 move at a first position in the same way as the distal section moved at the first position or a second position near the first position. In one or more additional or alternative embodiments, any other algorithm may be applied to control a continuum robot or catheter/probe, such as, but not limited to, Hold the Line, Close the Gap, Stay the Course, any combination thereof, etc.

Due to kinematics of a robot, device, or system embodiment of the present disclosure, applying a same “change in position” or a “change in state” to two separate orientations/states may maintain a difference (e.g., a set difference, a predetermined difference, etc.) between the two separate orientations/states. Since an orientation/state difference may be defined as the difference between wire position/state in one or more embodiments (other embodiments are not limited thereto), changing both sets of wire positions or states by the same amount would not affect the orientation or state difference between the two separate orientations or states.

Orientations mapped to two subsequent stage positions/states (or positions/states of another structure used for mapping path or path-like information) may have a specific orientation difference between the orientations. In a case where smoothing is applied, the later (or second) stage position/state (or position/state of the another structure) has a same change in orientation that the earlier (or first) stage position/state (or position/state of the another structure) received such that the pose/state difference did not change. The smoothing process may include an additional step of a “small motion”, which operates to cause the pose/state difference to change by an amount of that small motion. Since the “small motion” operates to produce the same orientation/state change regardless of prior orientation/state, the small motion step operates to direct that orientation/state in a table towards a proper (e.g., set, desired, predetermined, selected, etc.) direction, while also maintaining a semblance or configuration of the prior path/state before the smoothing process was applied. Therefore, in one or more embodiments, it may be most efficient and effective to combine and compare wire positions or states to or with prior orientations or states while using a smoothing process to maintain the pre-existing orientation changes.

In one or more embodiments, a catheter or probe may transition, move, or adjust using a shortest possible volume. In a case where a following section or portion of the probe or catheter is being transitioned, moved, or adjusted, using the shortest possible volume my reduce or minimize an amount of disruption to positions or states of one or more (or all) of the distal/following sections or portions of the catheter or probe. In one or more embodiments, a process or algorithm may perform the transitioning, moving, or adjusting process more efficiently than computing a transformation stackup of each section or portion of the catheter or probe. Preferably, each interpolated step aims towards the final orientation in a desired direction such that any prior orientation which the interpolated step is combined with will also aim towards the desired direction to achieve the final orientation.

In one or more embodiments of the present disclosure, an apparatus or system may include one or more processors that operate to: receive or obtain an image or images showing pose or position (or other state) information of a tip section of a catheter or probe having a plurality of sections including at least the tip section; track a history of the pose or position (or other state) information of the tip section of the catheter or probe during a period of time; and use the history of the pose or position (or other state) information of the tip section to determine how to align or transition, move, or adjust (e.g., robotically, manually, automatically, etc.) each section of the plurality of sections of the catheter or probe.

In one or more embodiments, one or more additional image or images may be received or obtained to show the catheter or probe after each section of the plurality of sections of the catheter or probe has been aligned or adjusted (e.g., robotically, manually, automatically, etc.) based on the history of the pose or position (or other state) information of the tip section. In one or more embodiments, the apparatus or system may include a display to display the image or images showing the aligned or adjusted sections of the catheter or probe. In one or more embodiments, the pose or position (or other state) information may include, but is not limited to, a target pose or position (or other state) or a final pose or position (or other state) that the tip section is set to reach, an interpolated pose or position (or other state) of the tip section (e.g., an interpolation of the tip section between two positions or poses (or other states) (e.g., between pose or position (or other state) A to pose or position (or other state) B) where the apparatus or system sends pose (or other state) change information in steps based on a desired, set, or predetermined speed; between poses or positions where each pose or position (or other state) of the catheter or probe takes or is disposed is tracked during the transition; etc.), and a measured pose or position (or other state) (e.g., using tracked poses or positions (or other states), using encoder positions (or other states) of each wire motor, etc.) where the one or more processors may further operate to calculate or derive a current position or position (or state) that a section (e.g., the tip section, one of the other sections of the plurality of sections of the probe or catheter, etc.) of the probe or catheter is taking. In addition to using one or more types of poses or positions (or other states), each pose or position (or state) may be converted (e.g., via the one or more processors) between the following formats: Drive Wire Positions (or state(s)); and/or Coordinates (three-dimensional (3D) Position and Orientation (or other state(s))).

In one or more embodiments, an apparatus or system may include a camera deployed at a tip of a catheter or probe and may be bent with the catheter or probe, and/or the camera may be detachably attached to, or removably inserted into, the steerable catheter or probe. In one or more embodiments, an apparatus or system may include a display controller, or the one or more processors may display the image or images for display on a display.

In the following embodiments, configurations are described that functionally interact with a flexible endoscope during an endoscopy procedure with imaging modalities including, for example, CT (computed tomography), MRI (magnetic resonance imaging), NIRF (near infrared fluorescence), NIRAF (near infrared auto-fluorescence), OCT (optical coherence tomography), SEE (spectrally encoded endoscope), IVUS (intravascular ultrasound), PET (positron emission tomography), X-ray imaging, combinations or hybrids thereof, other imaging modalities discussed herein, any combination thereof, or any modality known to those skilled in the art.

According to some embodiments, configurations are described as a robotic bronchoscope (or other scope, such as, but not limited to, an endoscope, other scopes discussed herein, or known to those skilled in the art) arrangement or a continuum robot arrangement that may be equipped with a tool channel for an imaging device and medical tools, where the imaging device and the medical tools may be exchanged by inserting and retracting the imaging device and/or the medical tools via the tool channel (see e.g., tool channel 126 in FIGS. 1-2, see e.g., imaging device or tool (e.g., camera) 160, and see e.g., medical tool 133 in FIG. 1). The imaging device can be a camera or other imaging device, and the medical tool can be a biopsy tool or other medical device. Configurations may facilitate placement of medical tools, catheters, needles or the like, and may be free standing, cart mounted, patient mounted, movably mounted, combinations thereof, or the like. The present disclosure is not limited to any particular configuration.

The robotic catheter or steerable catheter arrangement may be used in association with one or more displays and control devices and/or processors, such as those discussed herein (see e.g., one or more device or system configurations shown in one or more of FIGS. 1-15 of the present disclosure).

In one or more embodiments, the display device may display, on a monitor, an image captured by the imaging device, and the display device may have a display coordinate used for displaying the captured image. For example, top, bottom, right, and left portions of the monitor(s) or display(s) may be defined by axes of the displaying coordinate system/grid, and a relative position of the captured image or images against the monitor may be defined on the displaying coordinate system/grid.

The robotic catheter or scope arrangement may use one or more imaging devices (e.g., a catheter or probe 104, a camera, a sensor, any other imaging device discussed herein, etc.) and one or more display devices (e.g., a display 101-1, a display 101-2, a screen 1209, any other display discussed herein, etc.) to facilitate viewing, imaging, and/or characterizing tissue, a sample, or other object using one or a combination of the imaging modalities described herein.

In addition, a control device or a portion of a catheter, imaging device, robotic catheter, etc. (e.g., an actuator, one or more processors, one or more driving features, a motor, any combination thereof, etc.) may control a moving direction of the tool channel (e.g., the tool channel 126) or the camera (e.g., the camera 180). For example, the tool channel or the camera may be bent according to a control by the system (such as, but not limited to, the features discussed herein and shown in at least FIGS. 3A-3D). The system may have an operational controller (for example, a gamepad, a joystick 105 (see e.g., FIGS. 1-2), etc.) and a control coordinate. The control coordinate system/grid may define a moving (or bending) direction of the tool channel or the camera in one or more embodiments, including, but not limited to, in a case where a particular command is input by the operational controller. For example, in a case where a user inputs an “up” command via the operational controller, then the tool channel or the camera moves toward a direction which is defined by the control coordinate system/grid as an upward direction.

Before a user operates the robotic catheter or a catheter or probe 104 of any of the systems discussed herein, a calibration may be performed. By the calibration, a direction to which the tool channel or the camera moves or is bent according to a particular command (up, down, turn right, or turn left; alternatively, a command set may include a first direction, a second direction opposite or substantially opposite from or to the first direction, a third direction that is about or is 90 degrees from or to the first direction, and a fourth direction that is opposite or substantially opposite from or to the third direction) is adjusted to match a direction (top, bottom, right or left) on a display (or on the display coordinate).

For example, the calibration is performed so that an upward of the displayed image on the display coordinate corresponds to an upward direction on the control coordinate (a direction to which the tool channel or the camera moves according to an “up” command). Additionally or alternatively, first, second, third, and fourth directions on the display correspond to the first, second, third, and fourth directions of the control coordinate (e.g., of the tool channel or camera).

By the calibration, when a user inputs an “up” or a first direction command of the tool channel or the camera, the tool channel or the camera is bent to an upward or first direction on the control coordinate. The direction to which the tool channel or the camera is bent corresponds to an upward or first direction of the capture image displayed on the display.

In addition, a rotation function of a display of the captured image on the display coordination may be performed. For example, when the camera is deployed, the orientation of the camera view (top, bottom, right, and/or left) should match with a conventional orientation of the camera view that physicians or other medical personnel typically see in their normal catheter, imaging device, scope, etc. procedure: for example, for a bronchoscope, the right and left main bronchus may be displayed horizontally on a monitor or display (e.g., the display 101-1, the display 101-2, the display or screen 1209, etc.). Then, if the right and left main bronchus in a captured image are not displayed horizontally on the display, a user may rotate the captured image on the display coordinate so that the right and left main bronchus are displayed horizontally on the monitor or display (e.g., the display 101-1, the display 101-2, the display or screen 1209, etc.).

If the captured image is rotated on the display coordinate after a calibration is performed, a relationship between the top, bottom, right, and left (or first, second, third, and/or fourth directions) of the displayed image and top, bottom, right, and left (or corresponding first, second, third, and/or fourth directions) of the monitor may be changed. On the other hand, the tool channel or the camera may move or may be bent in the same way regardless of the rotation of the displayed image when a particular command is received (for example, a command to let the tool channel or the camera (or a capturing direction of the camera) move upward, downward, right, or left or to move in the first direction, second direction, third direction, or fourth direction). In one or more embodiments, a calibration or arrangement of the imaging device, scope, catheter or probe, may use an orientation feature 406 discussed below.

This causes a change of a relationship between the top, bottom, right, and left (or first, second, third, and fourth directions) of the monitor and a direction to which the tool channel or the camera moves (up, down, right, or left; or a first, second, third, or fourth direction) on the monitor according to a particular command (for example, tilting a joystick to up, down, right, or left; tilting the joystick in a first direction, the second direction, the third direction, or the fourth direction; etc.). For example, when the calibration is performed, by tilting the joystick upward (or to a first direction), the tool channel or the camera is bent to a direction corresponding to a direction top (or to the first direction) of or on the monitor. However, after the captured image on the display is rotated, by tilting the joystick upward (or to the first direction), the tool channel or the camera may not be bent to the direction corresponding to the direction of the top (or of the first direction) of the monitor but may be bent to a direction to a diagonally upward of the monitor. This may complicate user interaction.

When the camera is inserted into a continuum robot or steerable catheter apparatus or system or any other system or apparatus discussed herein, an operator may map or calibrate the orientation of the camera view, the user interface device, and the robot end-effector. However, this may not be enough for bronchoscopists, in one or more situations, because (1) the right and left main bronchus may be displayed in arbitrary direction in this case, and (2) bronchoscopists rely on how the bronchi look to navigate a bronchoscope and bronchoscopists typically confirm the location of the bronchoscope using or based on how the right and left main bronchus look like.

According to some embodiments, a direction to which a tool channel or a camera moves or is bent is corrected automatically in a case where a displayed image is rotated. The robot configurational embodiments described below enable to keep a correspondence between a direction on a monitor (top, bottom, right, or left of the monitor; a first, second, third, or fourth direction(s) of the monitor, etc.), a direction the tool channel or the camera moves on the monitor or display (e.g., the display 101-1, the display 101-2, the display or screen 1209, etc.) according to a particular directional command (up, down, turn right, or turn left; first direction, second direction, third direction, or fourth direction, etc.), and a user interface device even in a case where the displayed image is rotated. In one or more embodiments, there may be more than four directions set or corresponding between the monitor or display (e.g., the display 101-1, the display 101-2, the display or screen 1209, etc.), the tool channel or camera, and/or the image display or user interface device.

In one or more embodiments, medical image processing implements functioning through use of one or more processes, techniques, algorithms, or other steps discussed herein, that operate to provide rapid, accurate, cost-effective, and minimally invasive structure and manufacture/use techniques for structure of a catheter and/or a catheter tip.

In the present disclosure, one or more configurations are described that find use in therapeutic or diagnostic procedures in anatomical regions including the respiratory system, the digestive system, the bronchus, the lung, the liver, esophagus, stomach, colon, urinary tract, or other areas.

A medical apparatus or system according to one or more embodiments provides advantageous features to continuum robots or steerable catheters by providing rapid, accurate, cost-effective, and minimally invasive structure and manufacture/use techniques for structure of a catheter and/or a catheter tip and providing work efficiency to physicians during a medical procedure and rapid, accurate, and minimally invasive techniques for patients.

Referring back to FIG. 1, a medical apparatus or system 1000 may be provided in the form of a continuum robot or steerable catheter (or other imaging device) assembly or configuration that provides medical imaging with rapid, accurate, cost-effective, and minimally invasive structure and manufacture/use techniques for structure of a catheter and/or a catheter tip according to one or more embodiments. FIGS. 2-4 and 10 show one or more hardware configurations of the system 1000 as discussed above for FIG. 1. The system 1000 (or any other system discussed herein) may include one or more medical tools 133 and one or more medical devices or catheters/probes 104 (see e.g., as shown in FIG. 1). The medical tool 133 may be referred to as a “biopsy tool”, a “medical procedure tool”, an “imaging tool”, etc. in one or more embodiments and the medical device 104 is referred to as a “catheter” (or probe, continuum robot, steerable catheter, etc.). That said, the medical tool 133 and the medical device 104 are not limited thereto, and a variety of other types of tools, devices, configurations, or arrangements also falls within the scope of the present disclosure, including, but not limited to, for example, a bronchoscope, catheter, robotic bronchoscope, robotic catheter, endoscope, colonoscope, ablation device, sheath, guidewire, needle, probe, forceps, another medical tool, a camera, an imaging device, etc.

The controller or joystick 105 may have a housing with an elongated handle or handle section which may be manually grasped, and one or more input devices including, for example, a lever or a button or another input device that allows a user, such as a physician, nurse, technician, etc., to send a command to the medical apparatus or system 1000 (or any other system or apparatus discussed herein) to move the catheter 104. The controller or joystick 105 may execute software, computer instructions, algorithms, etc., so the user may complete all operations with the hand-held controller 105 by holding it with one hand, and/or the controller or joystick 105 may operate to communicate with one or more processors or controllers (e.g., processor 1200, controller 102, display controller 100, any other processor, computer, or controller discussed herein or known to those skilled in the art, etc.) that operate to execute software, computer instructions, algorithms, methods, other features, etc., so the user may complete any and/or all operations.

As aforementioned, the medical device 104 may be configured as or operate as a bronchoscope, catheter, endoscope, or another type of medical device. The system 1000 (or any other system discussed herein) may use an imaging device, where the imaging device may be a mechanical, digital, or electronic device configured to record, store, or transmit visual images, e.g., a camera, a camcorder, a motion picture camera, etc.

The display controller 100, the controller 102, a processor (such as, but not limited to, the processor 1200, any other processor discussed herein, etc.), etc. may operate to execute software, computer instructions, algorithms, methods, etc., and control a display of a navigation screen on the display 101-1, other types of imagery or information on the mini-display or other display 101-2, a display on a screen 1209, etc. The display controller 100, the controller 102, a processor (such as, but not limited to, the processor 1200, any other processor discussed herein, etc.), etc. may generate a three-dimensional (3D) model of an internal branching structure, for example, lungs or other internal structures, of a patient based on medical images such as CT, MRI, another imaging modality, etc. Additionally or alternatively, the 3D model may be received by the display controller 100, the controller 102, a processor (such as, but not limited to, the processor 1200, any other processor discussed herein, etc.), etc. from another device. The display controller 100, the controller 102, a processor (such as, but not limited to, the processor 1200, any other processor discussed herein, etc.), etc. may acquire catheter position information from the tracking sensor 106 (e.g., an electromagnetic (EM) tracking sensor) and/or from the catheter tip position/orientation/pose/state detector 107. The display controller 100, the controller 102, a processor (such as, but not limited to, the processor 1200, any other processor discussed herein, etc.), etc. may generate and output a navigation screen to any of the displays 101-1, 101-2, 1209, etc. based on the 3D model and the catheter position information by executing the software and/or by performing one or more algorithms, methods, and/or other features of the present disclosure. One or more of the displays 101-1, 101-2, 1209, etc. may display a current position of the catheter 103 on the 3D model, and/or the display controller 100, the controller 102, a processor (such as, but not limited to, the processor 1200, any other processor discussed herein, etc.), etc. may execute a correction of the acquired 3D model based on the catheter position information so as to minimize a divergence between the catheter position and a path mapped out on the 3D model.

The display controller 100, the controller 102, a processor (such as, but not limited to, the processor 1200, any other processor discussed herein, etc.), etc. and/or any console thereof may include one or more or a combination of levers, keys, buttons, switches, a mouse, a keyboard, etc., to control the elements of the system 1000 (or any other system or apparatus discussed herein) and each may have configurational components, as shown in FIGS. 4 and 10 as aforementioned, and may include other elements or components as discussed herein or known to those skilled in the art. The components of the system 1000 (or any other apparatus or system discussed herein) may be interconnected with medical instruments or a variety of other devices, and may be controlled independently, externally, or remotely by the display controller 100, the controller 102, a processor (such as, but not limited to, the processor 1200, any other processor discussed herein, etc.), etc.

A sensor, such as, but not limited to, the tracking sensor 106, a tip position detector 107, any other sensor discussed herein, etc. may monitor, measure or detect various types of data of the system 1000 (or any other apparatus or system discussed herein), and may transmit or send the sensor readings or data to a host through a network. The I/O interface or communication 1205 may interconnect various components with the medical apparatus or system 1000 to transfer data or information, or facilitate communication, to or from the apparatus or system 1000.

A power source may be used to provide power to the medical apparatus or system 1000 (or any other apparatus or system discussed herein) to maintain a regulated power supply, and may operate in a power-on mode, a power-off mode, and/or other modes. The power source may include or comprise a battery contained or included in the medical apparatus or system 1000 (or other apparatus or system discussed herein) and/or may include an external power source such as line power or AC power from a power outlet that may interconnect with the medical apparatus or system 1000 (or other system or apparatus of the present disclosure) through an AC/DC adapter and a DC/DC converter, or an AC/DC converter (or using any other configuration discussed herein or known to those skilled in the art) in order to adapt the power voltage from a source into one or more voltages used by components in the medical apparatus or system 1000 (and/or any other system or apparatus discussed herein).

Any of the sensors or detectors discussed herein, including, but not limited to, the sensor 106, the detector 107, etc. may include one or more or a combination of a processor, detection circuitry, memory, hardware, software, firmware, and may include other circuitry, elements, or components. Any of the sensors or detectors discussed herein, including, but not limited to, the sensor 106, the detector 107, etc. may be a plurality of sensors and may acquire sensor information output from one or more sensors that detect force, motion, current position and movement of components interconnected with the medical apparatus or system 1000 (or any other apparatus or system of the present disclosure). Any of the sensors or detectors discussed herein, including, but not limited to, the sensor 106, the detector 107, etc. may include a multi-axis acceleration or accelerometer sensor and a multi-axis gyroscope sensor, may be a combination of an acceleration and gyroscope sensors, may include other sensors, and may be configured through the use of a piezoelectric transducer, a mechanical switch, a single axis accelerometer, a multi-axis accelerometer, or other types of configurations. Any of the sensors or detectors discussed herein, including, but not limited to, the sensor 106, the detector 107, etc. may monitor, detect, measure, record, or store physical, operational, quantifiable data or other characteristic parameters of the medical apparatus or system 1000 (or any other system or apparatus discussed herein) including one or more or a combination of a force, impact, shock, drop, fall, movement, acceleration, deceleration, velocity, rotation, temperature, pressure position, orientation, motion, or other types of data of the medical apparatus or system 1000 (and/or other apparatus or system discussed herein) in multiple axes, in a multi-dimensional manner, along an x axis, y axis, z axis, or any combination thereof, and may generate sensor readings, information, data, a digital signal, an electronic signal, or other types of information corresponding to the detected state.

The medical apparatus or system 1000 may transmit or send the sensor reading data wirelessly or in a wired manner to a remote host or server. Any of the sensors or detectors discussed herein, including, but not limited to, the sensor 106, the detector 107, etc. may be interrogated and may generate a sensor reading signal or information that may be processed in real time, stored, post processed at a later time, or combinations thereof. The information or data that is generated by any of the sensors or detectors discussed herein, including, but not limited to, the sensor 106, the detector 107, etc. may be processed, demodulated, filtered, or conditioned to remove noise or other types of signals. Any of the sensors or detectors discussed herein, including, but not limited to, the sensor 106, the detector 107, etc. may include one or more or a combination of a force sensor, an acceleration, deceleration, or accelerometer sensor, a gyroscope sensor, a power sensor, a battery sensor, a proximity sensor, a motion sensor, a position sensor, a rotation sensor, a magnetic sensor, a barometric sensor, an illumination sensor, a pressure sensor, an angular position sensor, a temperature sensor, an altimeter sensor, an infrared sensor, a sound sensor, an air monitoring sensor, a piezoelectric sensor, a strain gauge sensor, a sound sensor, a vibration sensor, a depth sensor, and may include other types of sensors.

The acceleration sensor, for example, may sense or measure the displacement of mass of a component of the medical apparatus or system 1000 with a position or sense the speed of a motion of the component of the medical apparatus or system 1000 (or other apparatus or system). The gyroscope sensor may sense or measure angular velocity or an angle of motion and may measure movement of the medical apparatus or system 1000 in up to six total degrees of freedom in three-dimensional space including three degrees of translation freedom along cartesian x, y, and z coordinates and orientation changes between those axes through rotation along one or more or of a yaw axis, a pitch axis, a roll axis, and a horizontal axis. Yaw is when the component of the medical apparatus or system 1000 (or other apparatus or system) twists left or right on a vertical axis. Rotation on the front-to-back axis is called roll. Rotation from side to side is called pitch.

The acceleration sensor may include, for example, a gravity sensor, a drop detection sensor, etc. The gyroscope sensor may include an angular velocity sensor, a hand-shake correction sensor, a geomagnetism sensor, etc. The position sensor may be a global positioning system (GPS) sensor that receives data output from a GPS. The longitudinal and latitude of a current position may be obtained from access points of a radio frequency identification device (RFID) and a WiFi device and information output from wireless base stations, for example, so that these detections may be used as position sensors. These sensors may be arranged internally or externally of the medical apparatus or system 1000 (or any other system or apparatus of the present disclosure).

The medical device 104, in one or more embodiments, may be configured as a catheter 104 as aforementioned and as shown in FIGS. 1-4, and may move based on any of the aforementioned algorithms, including, but not limited to, the FTL algorithm, the RFTL algorithm, the Hold the Line algorithm, the Bridge the Gap algorithm, the Stay the Course algorithm, any other algorithm known to those skilled in the art, etc. For example, by applying the FTL algorithm, the middle section and the proximal section (following sections) of the catheter 104 may move at a first position in the same way as the distal section moved at the first position or a second position near the first position.

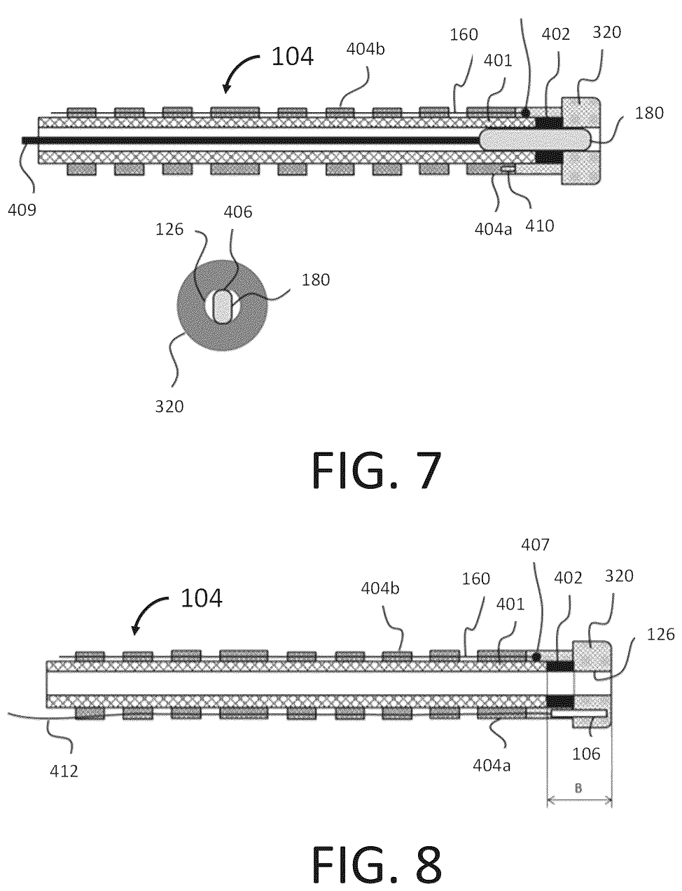

The display controller 100, the controller 102, a processor (such as, but not limited to, the processor 1200, any other processor discussed herein, etc.), etc. may operate to cause the catheter 104 to be placed in a pathway of a an object, target, or sample (e.g., a lung, an organ, a patient, tissue, etc.). The display controller 100, the controller 102, a processor (such as, but not limited to, the processor 1200, any other processor discussed herein, etc.), etc. may be configured to control a continuum robot or steerable catheter having an atraumatic tip, such as the catheter having an atraumatic tip 320 shown in FIG. 6, FIG. 7, and/or FIG. 8.

In one or more embodiments of the present disclosure, an apparatus may include: a catheter (e.g., the catheter 104), and an atraumatic tip attached to the distal portion of the catheter (e.g., the atraumatic tip 320 as shown in FIG. 6, FIG. 7, and/or FIG. 8). In one or more embodiments, the atraumatic tip 320 may be thermally attached to the distal portion of the catheter 104. The atraumatic tip 320 may be designed to include all critical geometry and/or structure related to catheter function, and the geometry and/or structure may operate to be preserved through a manufacturing process(es). In one or more embodiments, the atraumatic tip 320 may be a piece molded from a reflowable polymer material. The atraumatic tip 320 may have a counterbore that operates to accommodate a capture piece and/or a distal end of a braided shaft. Additional geometry for alignment or continuity may be added as needed. For example, additional extruded bosses on a proximal side of the atraumatic tip 320 may key into multi-lumens on a drive ring anchor of the catheter 104. The atraumatic tip 320 may have a durometer, and, while not limited thereto, the durometer may be one of the following: 35 D, 55 D, 63 D, 72 D, and/or a value in a range of 35 D-72 D. In one or more embodiments, the atraumatic tip 320 may be radiopaque, and the atraumatic tip 320 may sit flush with a distal drive ring or guide (e.g., drive ring or guide 404a shown in FIG. 6, FIG. 7, and/or FIG. 8) of the apparatus or system (e.g., the apparatus or system 1000, any other apparatus or system discussed herein).

The atraumatic tip 320 may include a tip piece 320 and a capture piece 402 (e.g., as shown in FIG. 6, FIG. 7, and/or FIG. 8), where the tip piece 320 may be attached to the capture piece 402 that operates to capture (or fully capture) a braided inner cover or shaft 401 of the catheter 104. The capture piece 402 may be made of a reflowable polymer material, compatible with the braided inner cover or shaft 401. The polymer material for the capture piece 402 may be Pebax (or any other block copolymer variation of PEBA (polyether block amide)). The capture piece 402 may be made of higher durometer than the atraumatic tip piece 320 so that the higher durometer may provide useful structure for the point and may preserve the captured ends of the braided inner cover or shaft 401 during later reflow processes of the tip piece 320 of the atraumatic tip. In one or more embodiments, the capture piece 402 may be, or may also be, radiopaque. RF stripes, radiopaque stripes, or another alternative color stripe may be useful for indicating the orientation of the atraumatic tip 320 and capture during the attachment process. The colored stripes may also aid in camera loading and docking into the anti-twist feature on a cap of the atraumatic tip 320. RF stripes or radiopaque stripes may be custom designed for this or any other purpose or feature discussed herein. The capture piece 402 operates to allow free wire ends of the braided inner cover or shaft 401 to be fully secured and anchored within a layer of polymer prior to being integrated into the tip piece of the atraumatic tip 320. The capture piece 402 on the braided inner cover or shaft may be seated into the tip piece of the atraumatic tip 320. The atraumatic tip piece 320 may be attached flush against the last drive or guide ring of the catheter 104, and/or the atraumatic tip piece 320 may be joined and flown together with the last distal drive ring or guide 404a through the thermal attachment process(es) or step(s).

In one or more embodiments of the present disclosure, an apparatus or system (e.g., the apparatus or system 1000, any other apparatus or system discussed herein) may include: a catheter 104 having at least a braided inner cover or shaft 401, a capture piece 402 attached to a distal end of the braided inner cover or shaft 401, and an atraumatic tip piece 320 attached with or to a distal end of the capture piece 402 (e.g., as shown in FIG. 6, FIG. 7, and/or FIG. 8). In one or more embodiments, the atraumatic tip piece 320 may bridge the capture piece 402 to the braided inner cover or shaft 401 (e.g., such that the atraumatic tip piece 320 may touch or contact the braided inner cover or shaft 401). The atraumatic tip piece 320 may include a counterbore to accommodate/bridge the capture piece 402 and the braided inner cover or shaft 401. The atraumatic tip piece 320 may include a tool channel 126, and the tool channel 126 may be connected to the inner diameter of the braided inner cover or shaft 401 continuously (e.g., the tool channel 126 may be co-axial with the inner diameter of the braided inner cover or shaft 401, the tool channel 126 may be in alignment with the inner diameter of the braided inner cover or shaft 401, the inner diameter of the braided inner cover or shaft 401 may match or substantially match the diameter of the tool channel 126 of the atraumatic tip piece 320, etc.). As described above, the capture piece 402 and/or the atraumatic tip piece 320 may be made of polymer, thermoplastic elastomer, or other similar material known to those skilled in the art. As aforementioned, the capture piece 402 and/or the atraumatic piece 320 may be made of a radiopaque material.

The drive or guide ring(s) (e.g., the drive or guide ring(s) 404) may be attached to the braided inner cover or shaft 402. Driving wires may be passively threaded through the lumens of the guide or drive ring(s) and may be terminated at a distal most guide or drive ring. The distal most guide or drive ring may be attached with or to the atraumatic tip piece. The top portion of FIG. 6 shows a cross-section side view of an embodiment of at least one catheter of the present disclosure. The bottom portion of FIG. 6 shows a front view of the catheter of a distal end for the catheter 104 of the top portion of FIG. 6. The atraumatic tip piece 320 may bridge the braided inner cover or shaft 401 and the capture piece 402 with a counter bore as shown in FIG. 6. The atraumatic tip piece may include a tool channel 126, and the tool channel 126 may be concatenated to the inner diameter (diameter A shown in FIG. 6) of the inner cover or shaft 401 continuously. The concatenated channel operates to provide access of tools from the proximal end of the catheter 104 to the distal end of the catheter 104. The atraumatic tip piece 320 may be attached to or with drive or guide rings 404. The drive or guide rings 404 may also be bonded on the braided inner cover or shaft 401 with intervals equidistantly (e.g., the drive or guide rings 404 or spaced out from each other by the set or predetermined equidistant intervals along the catheter 104). The driving wire 160 may be slideably held by the drive or guide rings 404 and may be terminated with or at an anchor 407 at the drive rings or guide located at the distalmost position of the catheter (see e.g., the drive or guide ring 404 positioned next to or closest to the atraumatic tip 320). The catheter 104 may bend by pulling and pushing the drive wire or wires 160 as discussed herein.

The top portion of FIG. 7 shows a cross-section side view of an embodiment of at least one catheter of the present disclosure. The bottom portion of FIG. 7 shows a front view of the catheter of a distal end for the catheter 104 of the top portion of FIG. 7. FIG. 7 shows a state of the catheter (e.g., the catheter 104) having a camera 180 therein. Camera 180 may include a camera shaft 409 and operates to capture an image, such as, but not limited to, an endoscopic image. The camera 180 may be detachably attached with the tool channel 126. In one or more embodiments, the atraumatic tip piece 320 may include one or more extruded or extruding bosses 410 that are inserted to, or operate to be inserted to, one or more respective conduits or acceptors 166 in the distal most guide or drive ring 404. As shown in the bottom part of FIG. 7 (showing the atraumatic tip piece 320 from a front view), the atraumatic tip piece 320 may include a tool channel 126 having or including an orientation feature 406 where a camera 180 may be detachably attached with, or removably inserted, into the tool channel 126. The orientation feature 406 may determine a rotational orientation of the camera 180 to the atraumatic tip piece 320, and the extruded boss or bosses 410 may determine a rotational orientation of the atraumatic tip 320 to the distal most guide or drive ring 404a (as shown in the top portion of FIG. 7). While not limited hereto, in the particular example shown in FIG. 7, the orientation feature 406 shows a flat orientation. The camera 180 has a flat orientation (or “orientation flat”) and operates to mate to or with the orientation feature 406. With the orientation feature 406, the camera 180 may have consistent rotational position, orientation, and/or state information with respect to the atraumatic tip piece 320 and may operate to suppress uncontrolled rotation or movement between the camera 180 and the atraumatic tip piece 320. The extruded boss(es) 410 may be inserted into a distalmost guide or drive ring 404a, and the extruded boss(es) 410 may determine a rotational position or state of the atraumatic tip piece 320 against or with respect to the distalmost guide or drive ring 404a. With the rotational positioning/adjusting with the extruded boss(es) 410, the atraumatic tip piece 320 may establish a designed, set, or predetermined rotational position, orientation, and/or state with respect to or against the driving wire layout with the guide or drive ring(s) 404. Therefore, in one or more embodiments, the camera 180 may establish consistent and designed, predetermined, or set rotational position, orientation, and/or state against, or with respect to, a bending coordinate of the catheter 104, even in cases where the camera 180 may be detachable and is attached/detached in the middle of a procedure or imaging by the operators of the catheter 104 and/or the apparatus(es), system(s), method(s), storage medium(s), and/or other features of the present disclosure.

FIG. 8 shows a cross sectional side view of at least one embodiment of a catheter (e.g., the catheter 104). The catheter 104 of FIG. 8 may have one or more of the features shown in FIGS. 6-7, and may also include a tracking sensor 106. In one or more embodiments, the catheter 104 may include a tracking sensor 106 that operates to measure a position and orientation (or other state information) of the distal end of the catheter 104. The tracking sensor 106 may be attached to or disposed in the atraumatic tip piece 320. The tracking sensor 106 may operate to locate an area from the distal end of the atraumatic tip piece 320 to the capture piece 402 (e.g., along a longitudinal direction, along a longitudinal axis of the catheter 104, in a direction co-linear or co-axial with the tool channel 126 of the atraumatic tip piece 320 and/or the inner diameter of the braided inner cover or shaft 401, etc.). The tracking sensor 106 may be an electromagnetic tracking sensor including a sensor coil. The tracking sensor 106 may include a tracking sensor cable 412. The tracking sensor cable 412 may run through the drive or guide rings 404 to the proximal end of the catheter 104. In one or more embodiments, a longitudinal position of the tracking sensor 106 in the atraumatic tip piece 320 may be in area B, which is from the distal end of atraumatic tip piece 320 to the proximal end of the capture piece 402, as shown in FIG. 8. By keeping the longitudinal position of the tracking sensor 106 within area B, the tracking sensor 106 may be away from the braided inner cover or shaft 401 while maintaining a robust and strong bonding among, between, or with the atraumatic tip piece 320, the braided inner cover or shaft 401, the capture piece 402, and the tracking sensor 106. Since the tracking sensor 106 (the EM sensor) may have interference in its measurement from metal objects, interference/error of the measurement of the tracking sensor 106 may be reduced by keeping the tracking sensor 106 away from metal braids in the braided inner cover or shaft 401.

In one or more embodiments of the present disclosure, a method of making a robotic catheter or an imaging apparatus may include: attaching a distal end of a braided inner cover or shaft of a catheter with or to a capture piece, and attaching an atraumatic tip piece to a distal end of the capture piece. The attachment steps may include or involve a thermal attachment process where at least the atraumatic tip piece is thermally attached to the catheter. The thermal attachment process may provide: a stronger overall joint than adhesive processes, a continuous inner lumen of the catheter, and/or all free wire ends of the braided inner cover or shaft may be fully captured and seated within the reflowed polymer (without risk of penetration to the inner lumen of the catheter). In one or more embodiments, the method may further include selecting a softer durometer material for a molded atraumatic tip piece as compared to the hardness of the capture piece to provide a configuration where: (i) the capture piece on the braided inner cover or shaft may remain intact while the molded atraumatic tip piece may be seated around the capture piece, (ii) free wire ends of the braided inner cover or shaft may not penetrate, or avoid penetration with, the inner or outer diameter of the catheter, and (iii) the wire ends are securely disposed or placed within a joint or bridge of the atraumatic tip piece that extends over and surrounds the capture piece of the catheter. In one or more embodiments, the method may further include applying or including radiopaque stripes, RF stripes, or other color stripes as visual aids on the catheter. RF stripes may be used for visualization under fluoroscopy (for example, while the apparatus or catheter is in use). Using added colorants or stripes may provide an advantage for visualization as the camera is loaded and passed through the inner lumen of the catheter. For example, use of colorants or stripes may allow for docking of the camera to an anti-twist feature in the atraumatic tip piece more seamlessly. In one or more embodiments, the method(s) may further include using a specific, set, or predetermined internal geometry and/or structure for the molded piece for the atraumatic tip piece so that the catheter remains intact, and the specific, set, or predetermined internal geometry and/or structure may include one or more of the following: (i) geometry and/or structure that operates to maintain or improve flow and vacuum suction rates; and/or (ii) geometry and/or structure that operates to achieve docking of the camera within the catheter. The method may further include using or adding extruded or extruding bosses on a proximal side of the atraumatic tip piece to align to the proximal skeleton or structure of the catheter (e.g., a robotic catheter), which may aid in the manufacturing process and provide consistent tip orientation between individual catheters. The method may further include disposing or incorporating a sensor (e.g., an electromagnetic (EM) sensor) within the geometry and/or structure of the distal tip and/or the atraumatic tip piece. The sensor may be used for tracking, and the EM sensor may be seated (or fully seated) within the distal tip or atraumatic tip piece and be protected by or within the distal tip piece. In one or more embodiments, the method may include fully integrating the sensor or EM sensor into the tip to better maintain or to achieve a smooth outer diameter on the catheter. The aforementioned geometrical and/or structural achievements of the manufacturing processes may be included in and possessed by the geometry and/or structure of any catheter of the present disclosure.

In one or more embodiments of the present disclosure, a storage medium stores instructions or a program for causing one or more processors of an apparatus or system to perform a method of manufacturing a robotic catheter or imaging apparatus, where the method may include: attaching a distal end of a braided inner cover or shaft of a catheter with or to a capture piece, and attaching an atraumatic tip piece to a distal end of the capture piece.

In one or more embodiments, an apparatus for performing navigation control and/or for controlling, manufacturing, or using a catheter and/or catheter tip may include a flexible medical device or tool; and one or more processors that operate to: bend a distal portion of the flexible medical device or tool; and advance the flexible medical device or tool through a pathway, wherein the flexible medical device or tool may be advanced through the pathway in a substantially centered manner.

In one or more embodiments, the flexible medical device or tool may have multiple bending sections, and the one or more processors may further operate to control or command the multiple bending sections of the flexible medical device or tool using one or more of the following modes: a Follow the Leader (FTL) mode, a Reverse Follow the Leader (RFTL) mode, a Hold the Line mode, a Close the Gap mode, and/or a Stay the Course mode. The flexible medical device or tool may include a catheter or scope and the catheter or scope may be part of, include, or be attached to an imaging apparatus, such as, but not limited to, an endoscope, a catheter, a probe, a bronchoscope, or any other imaging device discussed herein or known to those skilled in the art.

In one or more embodiments, a method for controlling an apparatus including a flexible medical device or tool that operates to perform navigation control and/or for controlling, manufacturing, or using a catheter and/or catheter tip may include: bending a distal portion of the flexible medical device or tool; and advancing the flexible medical device or tool through a pathway, wherein the flexible medical device or tool may be advanced through the pathway in a substantially centered manner. The flexible medical device or tool may have multiple bending sections, and the method may further include controlling or commanding the multiple bending sections of the flexible medical device or tool using one or more of the following modes: a Follow the Leader (FTL) process or mode, a Reserve Follow the Leader (RFTL) process or mode, a Hold the Line process or mode, a Close the Gap process or mode, and/or a Stay the Course process or mode.

In one or more embodiments, a non-transitory computer-readable storage medium storing at least one program for causing a computer to execute a method for controlling an apparatus including a flexible medical device or tool that operates to perform navigation control and/or for controlling, manufacturing, or using a catheter and/or catheter tip, where the method may include: bending a distal portion of the flexible medical device or tool; and advancing the flexible medical device or tool through a pathway, wherein the flexible medical device or tool may be advanced through the pathway in a substantially centered manner. The method may include any other feature discussed herein.

In a case where the medical device or catheter 104 includes a scope, the scope may comprise, for example, an anoscope, an arthroscope, a bronchoscope, a colonoscope, a colposcope, a cystoscope, an esophagoscope, a gastroscope, a laparoscope, a laryngoscope, a neuroendoscope, a proctoscope, a sigmoidoscope, a thoracoscope, an ureteroscope, or another device. In one or more embodiments, the scope preferably includes or comprises a bronchoscope.

Any units described throughout the present disclosure are merely for illustrative purposes and may operate as modules for implementing processes in one or more embodiments described in the present disclosure. However, one or more embodiments of the present disclosure are not limited thereto. The term “unit”, as used herein, may generally refer to firmware, software, hardware, or other component, such as circuitry, etc., or any combination thereof, that is used to effectuate a purpose. The modules may be hardware units (such as circuitry, firmware, a field programmable gate array, a digital signal processor, an application specific integrated circuit, any other hardware discussed herein or known to those skilled in the art, etc.) and/or software modules (such as a program, a computer readable program, instructions stored in a memory or storage medium, instructions downloaded from a remote memory or storage medium, other software discussed herein or known to those skilled in the art, etc.). Any units or modules for implementing one or more of the various steps discussed herein are not exhaustive or limited thereto. However, where there is a step of performing one or more processes, there may be a corresponding functional module or unit (implemented by hardware and/or software), or processor(s), controller(s), computer(s), etc. for implementing the one or more processes. Technical solutions by all combinations of steps described and units/modules/processors/controllers/etc. corresponding to these steps are included in the present disclosure.

In one or more embodiments, the medical apparatus or system 1000 of FIG. 1 may be configured as a continuum robot or steerable catheter arrangement with a multi-sectional catheter or probe configuration and follow the leader technology (or other control or movement, or tip, feature(s) and/or technique(s) discussed herein) to allow for precise catheter tip movement.

Additional features or aspects of present disclosure may also advantageously implement one or more AI (artificial intelligence) or machine learning algorithms, processes, techniques, or the like, to implement a method comprising: advancing the medical tool or catheter through a pathway and/or controlling a medical tool or catheter as discussed herein. Such AI techniques may use a neural network, a random forest algorithm, a cognitive computing system, a rules-based engine, other AI network structure discussed herein or known to those skilled in the art, etc., and are trained based on a set of data to assess types of data and generate output. For example, a training algorithm may be configured to implement a method comprising: advancing the medical tool or catheter through a pathway, wherein the medical tool or catheter is advanced through the pathway in a substantially centered manner. An AI-implemented algorithm may also be used to train a model to perform the manufacture and/or use of the continuum robot(s) and/or steerable catheter(s) having a tip piece as discussed herein.

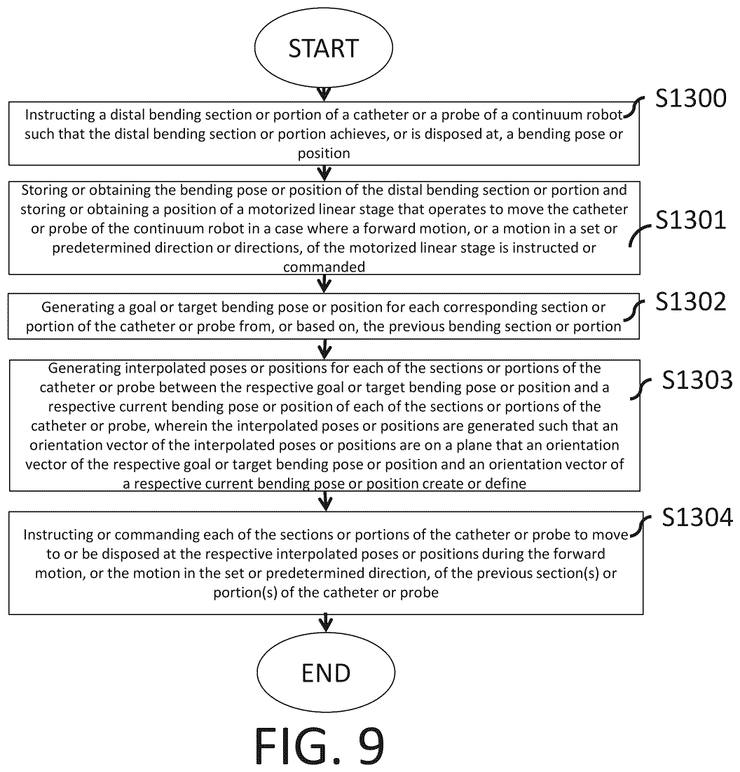

One or more features discussed herein may be used for performing control, correction, adjustment, and/or smoothing (e.g., direct FTL smoothing, path smoothing, continuum robot smoothing, etc.). FIG. 9 is a flowchart showing steps of at least one procedure for performing correction, adjustment, and/or smoothing of a continuum robot/catheter device (e.g., such as continuum robot/catheter device 104). One or more of the processors discussed herein may execute the steps shown in FIG. 9, and these steps may be performed by executing a software program read from a storage medium, including, but not limited to, the ROM110 or HDD 150, by CPU 120 or by any other processor discussed herein. One or more methods of performing correction, adjustment, and/or smoothing (e.g., direct FTL smoothing) for a catheter or probe of a continuum robot device or system may include one or more of the following steps: (i) in step S1300, instructing a distal bending section or portion of a catheter or a probe of a continuum robot such that the distal bending section or portion achieves, or is disposed at, a bending pose or position; (ii) in step S1301, storing or obtaining the bending pose or position of the distal bending section or portion and storing or obtaining a position of a motorized linear stage that operates to move the catheter or probe of the continuum robot in a case where a forward motion, or a motion in a set or predetermined direction or directions, of the motorized linear stage is instructed or commanded; (iii) in step S1302, generating a goal or target bending pose or position (or other state) for each corresponding section or portion of the catheter or probe from, or based on, the previous bending section or portion or based on a previous pose or state of a Distal bending section or portion; (iv) in step S1303, generating interpolated poses or positions for each of the sections or portions of the catheter or probe between the respective goal or target bending pose or position and a respective current bending pose or position of each of the sections or portions of the catheter or probe, wherein the interpolated poses or positions are generated such that an orientation vector of the interpolated poses or positions are on a plane that an orientation vector of the respective goal or target bending pose or position and an orientation vector of a respective current bending pose or position create or define; and/or (v) in step S1304, instructing or commanding each of the sections or portions of the catheter or probe to move to or be disposed at the respective interpolated poses or positions during the forward motion, or the motion in the set or predetermined direction, of the previous section(s) or portion(s) of the catheter or probe.

One or more of the aforementioned features may be used with a continuum robot and related features as disclosed in U.S. Provisional Pat. App. No. 63/150,859, filed on Feb. 18, 2021, the disclosure of which is incorporated by reference herein in its entirety, as disclosed in International Pat. App. No. PCT/US2022/016660, filed Feb. 16, 2022, the disclosure of which is incorporated by reference herein in its entirety, and as disclosed in U.S. patent application Ser. No. 17/673,606, filed Feb. 16, 2022, the disclosure of which is incorporated by reference herein in its entirety.

A user may provide an operation input through an input element or device, and the continuum robot apparatus or system 1000 may receive information of the input element and one or more input/output devices, which may include, but are not limited to, a receiver, a transmitter, a speaker, a display, an imaging sensor, a user input device, which may include a keyboard, a keypad, a mouse, a position tracked stylus, a position tracked probe, a foot switch, a microphone, etc. A guide device, component, or unit may include one or more buttons, knobs, switches, etc., that a user may use to adjust various parameters of the continuum robot 1000, such as the speed (e.g., rotational speed, translational speed, etc.), angle or plane, or other parameters.

The continuum robot apparatus 10 may be interconnected with medical instruments or a variety of other devices, and may be controlled independently, externally, or remotely a communication interface, such as, but not limited to the communication interface 1205. The communication interface 1205 may be configured as a circuit or other device for communicating with components included in the apparatus or system 1000, and with various external apparatuses connected to the apparatus via a network. For example, the communication interface 1205 may store information to be output in a transfer packet and may output the transfer packet to an external apparatus via the network by communication technology such as Transmission Control Protocol/Internet Protocol (TCP/IP). The apparatus may include a plurality of communication circuits according to a desired communication form. The CPU 1202, the communication interface 1205, and other components of the computer 1200 may interface with other elements including, for example, one or more of an external storage, a display, a keyboard, a mouse, a sensor, a microphone, a speaker, a projector, a scanner, a display, an illumination device, etc.

One or more control, adjustment, correction, and/or smoothing features of the present disclosure may be used with one or more image correction or adjustment features in one or more embodiments. One or more adjustments, corrections, or smoothing functions for a catheter or probe device and/or a continuum robot may adjust a path of one or more sections or portions of the catheter or probe device and/or the continuum robot (e.g., the continuum robot 104, the continuum robot device 10, etc.), and one or more embodiments may make a corresponding adjustment or correction to an image view. For example, in one or more embodiments the medical tool may be an endoscope, a bronchoscope, any other medical tool discussed herein, any other medical tool known to those skilled in the art, etc.

A computer, such as the console or computer 1200, may perform any of the steps, processes, and/or techniques discussed herein for any apparatus and/or system being manufactured or used, any of the embodiments shown in FIGS. 1-15, any other apparatus or system discussed herein, etc.

There are many ways to control a continuum robot, correct or adjust an image or a path (or one or more sections or portions of) a continuum robot (or other probe or catheter device or system), manufacture or use a catheter having a tip, or perform any other measurement or process discussed herein, to perform continuum robot method(s) or algorithm(s), and/or to control at least one continuum robot device/apparatus, system and/or storage medium, digital as well as analog. In at least one embodiment, a computer, such as the console or computer 1200, may be dedicated to control and/or use continuum robot devices, systems, methods, and/or storage mediums for use therewith described herein.

The one or more detectors, sensors, cameras, or other components of the apparatus or system embodiments (e.g. of the system 1000 of FIG. 1 or any other system discussed herein) may transmit the digital or analog signals to a processor or a computer such as, but not limited to, an image processor or display controller 100, a controller 102, a CPU 1201, a processor or computer 1200 (see e.g., at least FIGS. 1-4 and 10-15), a combination thereof, etc. The image processor may be a dedicated image processor or a general purpose processor that is configured to process images. In at least one embodiment, the computer 1200, may be used in place of, or in addition to, the image processor or display controller 100 and/or the controller 102 (or any other processor or controller discussed herein, such as, but not limited to, the computer 1200, etc.). In an alternative embodiment, the image processor may include an ADC and receive analog signals from the one or more detectors or sensors of the system 1000 (or any other system discussed herein). The image processor may include one or more of a CPU, DSP, FPGA, ASIC, or some other processing circuitry. The image processor may include memory for storing image, data, and instructions. The image processor may generate one or more images based on the information provided by the one or more detectors, sensors, or cameras. A computer or processor discussed herein, such as, but not limited to, a processor of the devices, apparatuses or systems of FIGS. 1-4 and 10-15, the computer 1200, the image processor, etc. may also include one or more components further discussed herein below.

Electrical analog signals obtained from the output of the system 1000 or the components thereof, and/or from the devices, apparatuses, or systems of FIGS. 1-4 and 10-15, may be converted to digital signals to be analyzed with a computer, such as, but not limited to, the computers or controllers 100, 102 of FIG. 1, the computer 1200, etc.

As aforementioned, there are many ways to control a continuum robot, correct or adjust an image, correct, adjust, or smooth a path (or section or portion) of a continuum robot, manufacture or use a continuum robot or steerable catheter having a tip, or perform any other measurement or process discussed herein, to perform continuum robot method(s) or algorithm(s), and/or to control at least one continuum robot device/apparatus, system and/or storage medium, digital as well as analog. By way of a further example, in at least one embodiment, a computer, such as the computer or controllers 100, 102 of FIG. 1, the console or computer 1200, etc., may be dedicated to the control and the monitoring of the continuum robot devices, systems, methods and/or storage mediums described herein.

The electric signals used for imaging may be sent to one or more processors, such as, but not limited to, the processors or controllers 100, 102 of FIGS. 1-4, a computer 1200 (see e.g., FIG. 10, etc. as discussed further below, via cable(s) or wire(s), such as, but not limited to, the cable(s) or wire(s) 113 (see FIG. 1). Additionally or alternatively, the computers or processors discussed herein are interchangeable, and may operate to perform any of the feature(s) and method(s) discussed herein.

Various components of a computer system 1200 (see e.g., the console or computer 1200 as may be used as one embodiment example of the computer, processor, or controllers 100, 102 shown in FIG. 1) are provided in FIG. 10. A computer system 1200 may include a central processing unit (“CPU”) 1201, a ROM 1202, a RAM 1203, a communication interface 1205 (also referred as an Input/Output or I/O interface), a hard disk (and/or other storage device, such as, but not limited to, an SSD) 1204, a screen (or monitor interface) 1209, a keyboard (or input interface; may also include a mouse or other input device in addition to the keyboard) 1210 and a BUS (or “Bus”) or other connection lines (e.g., connection line 1213) between one or more of the aforementioned components (e.g., as shown in FIG. 10). In addition, the computer system 1200 may comprise one or more of the aforementioned components. For example, a computer system 1200 may include a CPU 1201, a RAM 1203, an input/output (I/O) interface (such as the communication interface 1205) and a bus (which may include one or more lines 1213 as a communication system between components of the computer system 1200; in one or more embodiments, the computer system 1200 and at least the CPU 1201 thereof may communicate with the one or more aforementioned components of a continuum robot device or system using same, such as, but not limited to, the system 1000, the devices/systems of FIGS. 1-4, and/or the systems/apparatuses or other components of FIG. 10, discussed herein above, via one or more lines 1213), and one or more other computer systems 1200 may include one or more combinations of the other aforementioned components (e.g., the one or more lines 1213 of the computer 1200 may connect to other components via line 113). The CPU 1201 is configured to read and perform computer-executable instructions stored in a storage medium. The computer-executable instructions may include those for the performance of the methods and/or calculations described herein. The computer system 1200 may include one or more additional processors in addition to CPU 1201, and such processors, including the CPU 1201, may be used for controlling and/or manufacturing a device, system, or storage medium for use with same or for use with any continuum robot technique(s), and/or use or manufacture catheter tip feature(s) and/or technique(s) discussed herein. The system 1200 may further include one or more processors connected via a network connection (e.g., via network 1206). The CPU 1201 and any additional processor being used by the system 1200 may be located in the same telecom network or in different telecom networks (e.g., performing, manufacturing, controlling, calculation, and/or using technique(s) may be controlled remotely).

The I/O or communication interface 1205 provides communication interfaces to input and output devices, which may include the one or more of the aforementioned components of any of the systems discussed herein (e.g., the controller 100, the controller 102, the displays 101-1, 101-2, the actuator 103, the continuum device 104, the operating portion or controller 105, the tracking sensor 106, the position detector 107, the rail 108, etc.), a microphone, a communication cable and a network (either wired or wireless), a keyboard 1210, a mouse, a touch screen or screen 1209, a light pen and so on. The communication interface of the computer 1200 may connect to other components discussed herein via line 113 (as diagrammatically shown in FIG. 10). The Monitor interface or screen 1209 provides communication interfaces thereto.

Any methods and/or data of the present disclosure, such as, but not limited to, the methods for using and/or controlling a continuum robot or catheter device, system, or storage medium for use with same and/or method(s) for imaging, performing tissue or sample characterization or analysis, performing diagnosis, planning and/or examination, for performing control or adjustment techniques (e.g., to a path of, to a pose or position of, or to one or more sections or portions of, a continuum robot, a catheter or a probe), for using or manufacturing catheter tip feature(s) and/or technique(s), and/or for performing image correction or adjustment or other technique(s), as discussed herein, may be stored on a computer-readable storage medium. A computer-readable and/or writable storage medium used commonly, such as, but not limited to, one or more of a hard disk (e.g., the hard disk 1204, a magnetic disk, etc.), a flash memory, a CD, an optical disc (e.g., a compact disc (“CD”) a digital versatile disc (“DVD”), a Blu-ray™ disc, etc.), a magneto-optical disk, a random-access memory (“RAM”) (such as the RAM 1203), a DRAM, a read only memory (“ROM”), a storage of distributed computing systems, a memory card, or the like (e.g., other semiconductor memory, such as, but not limited to, a non-volatile memory card, a solid state drive (SSD) (see storage 1204 may be an SSD instead of a hard disk in one or more embodiments; see also, storage 150 in FIG. 4), SRAM, etc.), an optional combination thereof, a server/database, etc. may be used to cause a processor, such as, the processor or CPU 1201 of the aforementioned computer system 1200 to perform the steps of the methods disclosed herein. The computer-readable storage medium may be a non-transitory computer-readable medium, and/or the computer-readable medium may comprise all computer-readable media, with the sole exception being a transitory, propagating signal in one or more embodiments. The computer-readable storage medium may include media that store information for predetermined, limited, or short period(s) of time and/or only in the presence of power, such as, but not limited to Random Access Memory (RAM), register memory, processor cache(s), etc. Embodiment(s) of the present disclosure may also be realized by a computer of a system or apparatus that reads out and executes computer executable instructions (e.g., one or more programs) recorded on a storage medium (which may also be referred to more fully as a “non-transitory computer-readable storage medium”) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s).

In accordance with at least one aspect of the present disclosure, the methods, devices, systems, and computer-readable storage mediums related to the processors, such as, but not limited to, the processor of the aforementioned computer 1200, the controller 100, the controller 102, etc., as described above may be achieved utilizing suitable hardware, such as that illustrated in the figures. Functionality of one or more aspects of the present disclosure may be achieved utilizing suitable hardware, such as that illustrated in FIG. 10. Such hardware may be implemented utilizing any of the known technologies, such as standard digital circuitry, any of the known processors that are operable to execute software and/or firmware programs, one or more programmable digital devices or systems, such as programmable read only memories (PROMs), programmable array logic devices (PALs), etc. The CPU 1200, 1201 (as shown in FIG. 10, and/or which may be included in the computer, processor, controller and/or CPU 100, 102, 1201, etc. of FIGS. 1-4 and FIGS. 10-15), etc. may also include and/or be made of one or more microprocessors, nanoprocessors, one or more graphics processing units (“GPUs”; also called a visual processing unit (“VPU”)), one or more Field Programmable Gate Arrays (“FPGAs”), or other types of processing components (e.g., application specific integrated circuit(s) (ASIC)). Still further, the various aspects of the present disclosure may be implemented by way of software and/or firmware program(s) that may be stored on suitable storage medium (e.g., computer-readable storage medium, hard drive, etc.) or media (such as floppy disk(s), memory chip(s), etc.) for transportability and/or distribution. The computer may include a network of separate computers or separate processors to read out and execute the computer executable instructions. The computer executable instructions may be provided to the computer, for example, from a network or the storage medium. The computers or processors (e.g., 100, 102, 1201, 1200, etc.) may include the aforementioned CPU structure, or may be connected to such CPU structure for communication therewith.

In one or more embodiments, a computer or processor may include an image/display processor or communicate with an image/display processor. For example, the computer 1200 includes a central processing unit (CPU) 1201, and may also include a graphical processing unit (GPU) 1215. Alternatively, the CPU 1201 or the GPU 1215 may be replaced by the field-programmable gate array (FPGA), the application-specific integrated circuit (ASIC) or other processing unit depending on the design of a computer, such as the computer 1200, controller or processor 100, controller or processor 102, any other computer, CPU, or processor discussed herein, etc.

At least one computer program is stored in the HDD/SSD 1204, the data storage 150, or any other storage device or drive discussed herein, and the CPU 1201 loads the at least one program onto the RAM 1203, and executes the instructions in the at least one program to perform one or more processes described herein, as well as the basic input, output, calculation, memory writing, and memory reading processes.

The computer, such as the computer 1200, the computer, processors, and/or controllers of FIGS. 1-4, FIG. 10, FIGS. 11-15, etc., communicates with the one or more components of the apparatuses/systems of FIGS. 1-4, of FIG. 10, and/or of any other apparatus(es) or system(s) discussed herein, to perform any of the methods, techniques, or features discussed herein, including, but not limited to, imaging, and may reconstruct an image from the acquired intensity data. The monitor or display 1209 displays the reconstructed image, and the monitor or display 1209 may display other information about the imaging condition or about an object to be imaged. The monitor 1209 also provides a graphical user interface for a user to operate a system, for example when performing CT, MRI, or other imaging modalities or other imaging technique(s), including, but not limited to, controlling continuum robot devices/systems, and/or manufacturing or using catheter tip feature(s) and/or technique(s). An operation signal is input from the operation unit (e.g., such as, but not limited to, a mouse device 1211, a keyboard 1210, a touch panel device, etc.) into the communication interface 1205 in the computer 1200, and corresponding to the operation signal the computer 1200 instructs the system (e.g., the system 1000, the systems/apparatuses of FIGS. 1-4 and 10, any other system/apparatus discussed herein, etc.) to start or end the imaging, and/or to start or end continuum robot control(s) and/or performance of correction, adjustment, and/or catheter tip feature(s) and/or technique(s). The camera or imaging device as aforementioned may have interfaces to communicate with the computer 1200 to send and receive the status information and the control signals.

As shown in FIG. 11, one or more processors or computers 1200, 1200′ (or any other processor discussed herein) may be part of a system in which the one or more processors or computers 1200, 1200′ (or any other processor discussed herein) communicate with other devices (e.g., a database 1603, a memory 1602 (which may be used with or replaced by any other type of memory discussed herein or known to those skilled in the art), an input device 1600, an output device 1601, etc.). In one or more embodiments, one or more models may have been trained previously and stored in one or more locations, such as, but not limited to, the memory 1602, the database 1603, etc. In one or more embodiments, it is possible that one or more models and/or data discussed herein (e.g., training data, testing data, validation data, imaging data, etc.) may be input or loaded via a device, such as the input device 1600. In one or more embodiments, a user may employ an input device 1600 (which may be a separate computer or processor, a keyboard such as the keyboard 1210, a mouse such as the mouse 1211, a microphone, a screen or display 1209 (e.g., a touch screen or display), or any other input device known to those skilled in the art). In one or more system embodiments, an input device 1600 may not be used (e.g., where user interaction is eliminated by one or more artificial intelligence features discussed herein). In one or more system embodiments, the output device 1601 may receive one or more outputs discussed herein to perform coregistration, autonomous navigation, movement detection, control, and/or any other process discussed herein. In one or more system embodiments, the database 1603 and/or the memory 1602 may have outputted information (e.g., trained model(s), detected marker information, image data, test data, validation data, training data, coregistration result(s), segmentation model information, object detection/regression model information, combination model information, etc.) stored therein. That said, one or more embodiments may include several types of data stores, memory, storage media, etc. as discussed above, and such storage media, memory, data stores, etc. may be stored locally or remotely.

Additionally, unless otherwise specified, the term “subset” of a corresponding set does not necessarily represent a proper subset and may be equal to the corresponding set.

While one or more embodiments of the present disclosure include various details regarding a neural network model architecture and optimization approach, in one or more embodiments, any other model architecture, machine learning algorithm, or optimization approach may be employed. One or more embodiments may utilize hyper-parameter combination(s). One or more embodiments may employ data capture, selection, annotation as well as model evaluation (e.g., computation of loss and validation metrics) since data may be domain and application specific. In one or more embodiments, the model architecture may be modified and optimized to address a variety of computer visions issues (discussed below).

One or more embodiments of the present disclosure may automatically detect (predict a spatial location of) a catheter tip (e.g., in or near an airway, pathway, a lung or other organ, in a patient, etc.) in a time series of X-ray images to co-register the X-ray images with the corresponding OCT images (at least one example of a reference point of two different coordinate systems). One or more embodiments may use deep (recurrent) convolutional neural network(s), which may improve catheter tip detection, tissue detection, tissue characterization, robotic control, and image co-registration significantly. One or more embodiments may employ segmentation and/or object/keypoint detection architectures to solve one or more computer vision issues in other domain areas in one or more applications. One or more embodiments employ several novel materials and methods to solve one or more computer vision or other issues (e.g., lesion detection in time series of X-ray images, for instance; tissue detection; tissue characterization; robotic control; etc.).

One or more embodiments employ data capture and selection. In one or more embodiments, the data is what makes such an application unique and distinguishes this application from other applications. For example, images may include a radiodense marker, a sensor (e.g., an EM sensor), or some other identifier that is specifically used in one or more procedures (e.g., used in catheters/probes with a similar marker, sensor, or identifier to that of an OCT marker, used in catheters/probes with a similar or same marker, sensor, or identifier even compared to another imaging modality, etc.) to facilitate computational detection of a marker, sensor, catheter, and/or tissue detection, characterization, validation, etc. in one or more images (e.g., X-ray images). One or more embodiments may couple a software device or features (model) to hardware (e.g., a robotic catheter or probe, a steerable probe/catheter using one or more sensors (or other identifier or tracking components), etc.). One or more embodiments may utilize animal data in addition to patient data. Training deep learning may use a large amount of data, which may be difficult to obtain from clinical studies. Inclusion of image data from pre-clinical studies in animals into a training set may improve model performance. Training and evaluation of a model may be highly data dependent (e.g., a way in which frames are selected (e.g., during steerable catheter control, frames obtained via a robotic catheter, etc.), split into training/validation/test sets, and grouped into batches as well as the order in which the frames, sets, and/or batches are presented to the model, any other data discussed herein, etc.). In one or more embodiments, such parameters may be more important or significant than some of the model hyper-parameters (e.g., batch size, number of convolution layers, any other hyper-parameter discussed herein, etc.). One or more embodiments may use a collection or collections of user annotations after introduction of a device/apparatus, system, and/or method(s) into a market, and may use post market surveillance, retraining of a model or models with new data collected (e.g., in clinical use), and/or a continuously adaptive algorithm/method(s).

One or more embodiments may employ data annotation. For example, one or more embodiments may label pixel(s) representing a marker, sensor, or identifier detection or a tissue and/or catheter detection, characterization, and/or validation as well as pixels representing a blood vessel(s) or portions of a pathway (e.g., a vessel, a lumen, an airway, a bronchial pathway, another type of pathway, etc.) at different phase(s) of a procedure/method (e.g., different levels of contrast due to intravascular contrast agent) of acquired frame(s).

One or more embodiments may employ incorporation of prior knowledge. For example, in one or more embodiments, a marker, sensor, or other portion of a robotic catheter/probe location may be known inside a vessel, pathway, airway, or bronchial pathway (or other type of pathway) and/or inside a catheter or probe; a tissue and/or catheter or catheter tip location may be known inside a vessel, an airway, a lung, a bronchial pathway, or other type of target, object, or specimen; etc. As such, simultaneous localization of the pathway, airway, bronchial pathway, lung, etc. and sensor(s)/marker(s)/identifier(s) may be used to improve sensor/marker detection and/or tissue and/or catheter detection, localization, characterization, and/or validation. For example, in a case where it is confirmed that the sensor of the probe or catheter, or the catheter or probe, is by or near a target area for tissue detection and characterization, the integrity of the tissue identification/detection and/or characterization for that target area is improved or maximized (as compared to a false positive where a tissue may be detected in an area where the probe or catheter (or sensor thereof) is not located). In one or more embodiments, a sensor or other portion of a catheter/probe may move inside a target, object, or specimen (e.g., a pathway, an airway, a bronchial pathway, a lung or another organ, in a patient, in tissue, etc.), and such prior knowledge may be incorporated into the machine learning algorithm or the loss function.

One or more embodiments may employ loss (cost) and evaluation function(s)/metric(s). For example, use of temporal information for model training and evaluation may be used in one or more embodiments. One or more embodiments may evaluate a distance between prediction and ground truth per frame as well as consider a trajectory of predictions across multiple frames of a time series.

### Application of Machine Learning

Application of machine learning may be used in one or more embodiment(s), as discussed in PCT/US2020/051615, filed on Sep. 18, 2020 and published as WO 2021/055837 A9 on Mar. 25, 2021, and as discussed in U.S. patent application Ser. No. 17/761,561, filed on Mar. 17, 2022, the applications and publications of which are incorporated by reference herein in their entireties. For example, at least one embodiment of an overall process of machine learning is shown below:
i. Create a dataset that contains both images and corresponding ground truth labels;
ii. Split the dataset into a training set and a testing set;
iii. Select a model architecture and other hyper-parameters;
iv. Train the model with the training set;
v. Evaluate the trained model with the validation set; and
vi. Repeat iv and v with new dataset(s).

Based on the testing results, steps i and iii may be revisited in one or more embodiments.

One or more models may be used in one or more embodiment(s) to detect and/or characterize a tissue or tissues and/or lesion(s), such as, but not limited to, the one or more models as discussed in PCT/US2020/051615, filed on Sep. 18, 2020 and published as WO 2021/055837 A9 on Mar. 25, 2021, and as discussed in U.S. patent application Ser. No. 17/761,561, filed on Mar. 17, 2022, the applications and publications of which are incorporated by reference herein in their entireties. For example, one or more embodiments may use a segmentation model, a regression model, a combination thereof, etc.

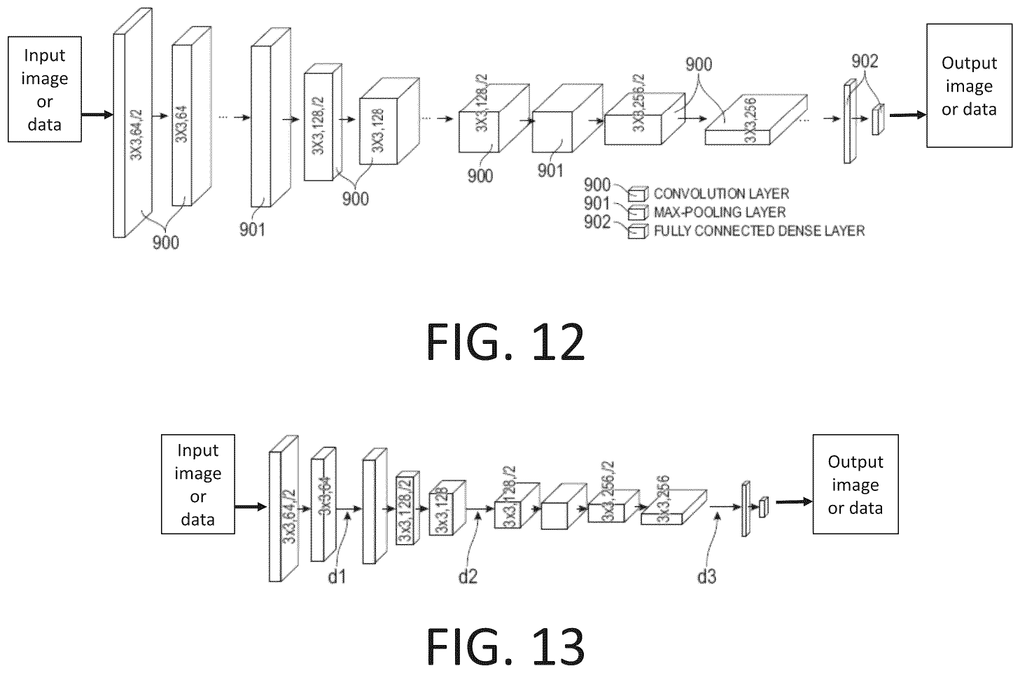

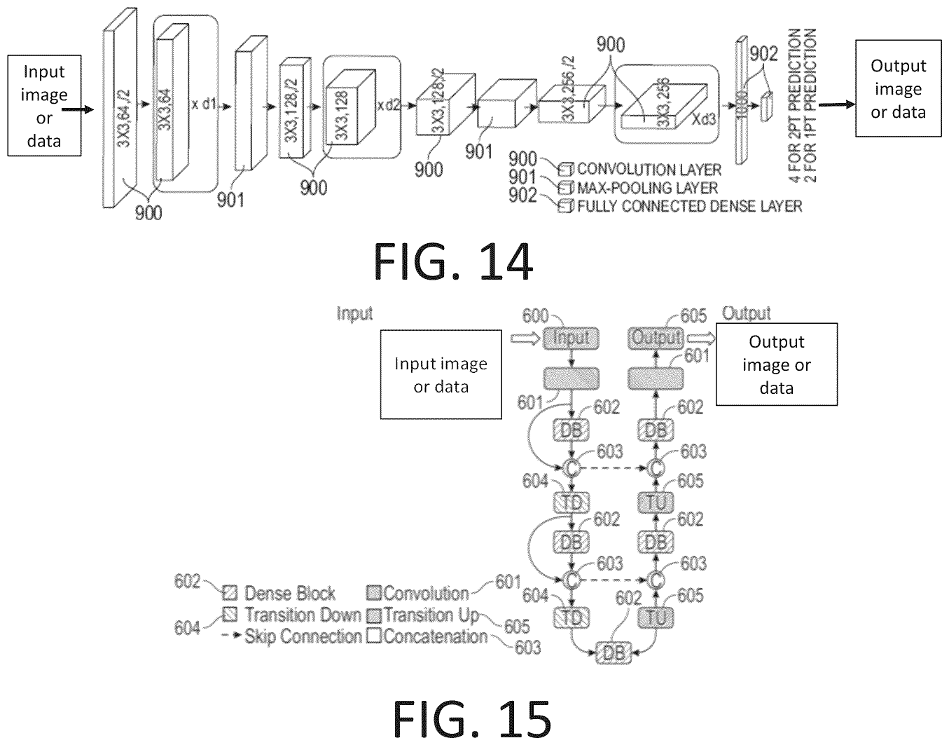

For regression model(s), the input may be the entire image frame or frames, and the output may be the centroid coordinates of sensors/markers (target sensor and stationary sensor or marker, if necessary/desired) and/or coordinates of a portion of a catheter or probe to be used in determining the localization and lesion and/or tissue detection and/or characterization. As shown diagrammatically in FIGS. 12-14, an example of an input image on the left side of FIGS. 12-14 and a corresponding output image on the right side of FIGS. 12-14 are illustrated for regression model(s). At least one architecture of a regression model is shown in FIG. 12. In at least the embodiment of FIG. 12, the regression model may use a combination of one or more convolution layers 900, one or more max-pooling layers 901, and one or more fully connected dense layers 902. While not limited to the Kernel size, Width/Number of filters (output size), and Stride sizes shown for each layer (e.g., in the left convolution layer of FIG. 12, the Kernel size is “3×3”, the Width/# of filters (output size) is “64”, and the Stride size is “2”). In one or more embodiments, another hyper-parameter search with a fixed optimizer and with a different width may be performed, and at least one embodiment example of a model architecture for a convolutional neural network for this scenario is shown in FIG. 13. One or more embodiments may use one or more features for a regression model as discussed in “Deep Residual Learning for Image Recognition” to Kaiming He, et al., Microsoft Research, Dec. 10, 2015 (https://arxiv.org/pdf/1512.03385.pdf), which is incorporated by reference herein in its entirety. FIG. 14 shows at least a further embodiment example of a created architecture of or for a regression model(s).

Since the output from a segmentation model, in one or more embodiments, is a “probability” of each pixel that may be categorized as a tissue or lesion characterization or tissue or lesion identification/determination, post-processing after prediction via the trained segmentation model may be developed to better define, determine, or locate the final coordinate of a tissue location, a catheter tip location, and/or a lesion location (or a sensor/marker location where the sensor/marker is a part of the catheter) and/or determine the type and/or characteristics of the tissue(s) and/or lesion(s). One or more embodiments of a semantic segmentation model may be performed using the One-Hundred Layers Tiramisu method discussed in “The One Hundred Layers Tiramisu: Fully Convolutional DenseNets for Semantic Segmentation” to Simon Jégou, et al., Montreal Institute for Learning Algorithms, published Oct. 31, 2017 (https://arxiv.org/pdf/1611.09326.pdf), which is incorporated by reference herein in its entirety. A segmentation model may be used in one or more embodiment, for example, as shown in FIG. 15. At least one embodiment may utilize an input 600 as shown to obtain an output 605 of at least one embodiment of a segmentation model method. For example, by applying the One-Hundred Layers Tiramisu method(s), one or more features, such as, but not limited to, convolution 601, concatenation 603, transition up 605, transition down 604, dense block 602, etc., may be employed by slicing the training data set. While not limited to only or by only these embodiment examples, in one or more embodiments, a slicing size may be one or more of the following: 100×100, 224×224, 512×512, and, in one or more of the experiments performed, a slicing size of 224×224 performed the best. A batch size (of images in a batch) may be one or more of the following: 2, 4, 8, 16, and, from the one or more experiments performed, a bigger batch size typically performs better (e.g., with greater accuracy). In one or more embodiments, 16 images/batch may be used. The optimization of all of these hyper-parameters depends on the size of the available data set as well as the available computer/computing resources; thus, once more data is available, different hyper-parameter values may be chosen. Additionally, in one or more embodiments, steps/epoch may be 100, and the epochs may be greater than (>) 1000. In one or more embodiments, a convolutional autoencoder (CAE) may be used.

In one or more embodiments, hyper-parameters may include, but are not limited to, one or more of the following: Depth (i.e., # of layers), Width (i.e., # of filters), Batch size (i.e., # of training images/step): May be >4 in one or more embodiments, Learning rate (i.e., a hyper-parameter that controls how fast the weights of a neural network (the coefficients of regression model) are adjusted with respect the loss gradient), Dropout (i.e., % of neurons (filters) that are dropped at each layer), and/or Optimizer: for example, Adam optimizer or Stochastic gradient descent (SGD) optimizer. In one or more embodiments, other hyper-parameters may be fixed or constant values, such as, but not limited to, for example, one or more of the following: Input size (e.g., 1024 pixel×1024 pixel, 512 pixel×512 pixel, another preset or predetermined number or value set, etc.), Epochs: 100, 200, 300, 400, 500, another preset or predetermined number, etc. (for additional training, iteration may be set as 3000 or higher), and/or Number of models trained with different hyper-parameter configurations (e.g., 10, 20, another preset or predetermined number, etc.).

One or more features discussed herein may be determined using a convolutional auto-encoder, Gaussian filters, Haralick features, and/or thickness or shape of the sample or object (e.g., the tissue or tissues, the lesion or lesions, the catheter or catheter tip, a lung or other organ, an airway, a bronchial pathway, another type of pathway, a specimen, a patient, a target in the patient, etc.).

One or more embodiments of the present disclosure may use machine learning to determine sensor, tissue, catheter or catheter tip, and/or lesion location; to determine, detect, or evaluate tissue and/or lesion type(s) and/or characteristic(s); and/or to perform any other feature discussed herein.

Machine learning (ML) is a field of computer science that gives processors the ability to learn, via artificial intelligence. Machine learning may involve one or more algorithms that allow processors or computers to learn from examples and to make predictions for new unseen data points. In one or more embodiments, such one or more algorithms may be stored as software or one or more programs in at least one memory or storage medium, and the software or one or more programs allow a processor or computer to carry out operation(s) of the processes described in the present disclosure. For example, machine learning may be used to train one or more models to efficiently to control a catheter or catheter tip, to manufacture or use the catheter or catheter tip, to perform imaging, etc.

The present disclosure and/or one or more components of devices, systems, and storage mediums, and/or methods, thereof also may be used in conjunction with continuum robot devices, systems, methods, and/or storage mediums and/or with endoscope devices, systems, methods, and/or storage mediums. Such continuum robot devices, systems, methods, and/or storage mediums are disclosed in at least: U.S. Provisional Pat. App. No. 63/150,859, filed on Feb. 18, 2021, the disclosure of which is incorporated by reference herein in its entirety. Such endoscope devices, systems, methods, and/or storage mediums are disclosed in at least: U.S. patent application Ser. No. 17/565,319, filed on Dec. 29, 2021, the disclosure of which is incorporated by reference herein in its entirety; U.S. Pat. App. No. 63/132,320, filed on Dec. 30, 2020, the disclosure of which is incorporated by reference herein in its entirety; U.S. patent application Ser. No. 17/564,534, filed on Dec. 29, 2021, the disclosure of which is incorporated by reference herein in its entirety; and U.S. Pat. App. No. 63/131,485, filed Dec. 29, 2020, the disclosure of which is incorporated by reference herein in its entirety. Any of the features of the present disclosure may be used in combination with any of the features as discussed in U.S. Prov. Pat. App. No. 63/378,017, filed Sep. 30, 2022, the disclosure of which is incorporated by reference herein in its entirety, as discussed in U.S. patent application Ser. No. 18/477,081, filed Sep. 28, 2023, the disclosure of which is incorporated by reference herein in its entirety, and/or any of the features as discussed in U.S. Prov. Pat. App. No. 63/377,983, filed Sep. 30, 2022, the disclosure of which is incorporated by reference herein in its entirety, and as discussed in U.S. Prov. Pat. App. No. 63/383,210, filed Nov. 10, 2022, the disclosure of which is incorporated by reference herein in its entirety. Any of the features of the present disclosure may be used in combination with any of the features as discussed in U.S. Pat. Pub. No. 2023/0131269, published on Apr. 26, 2023, the disclosure of which is incorporated by reference herein in its entirety. Any of the features of the present disclosure may be used in combination with any of the features a discussed in U.S. Pat. App. No. 63/587,637, filed on Oct. 3, 2023, the disclosure of which is incorporated by reference herein in its entirety, and/or as discussed in International Pat. App. No. PCT/US2024/037935, filed Jul. 12, 2024, the disclosure of which is incorporated by reference herein in its entirety.

Although the disclosure herein has been described with reference to particular embodiments, it is to be understood that these embodiments are merely illustrative of the principles and applications of the present disclosure (and are not limited thereto), and the present disclosure is not limited to the disclosed embodiments. It is therefore to be understood that numerous modifications may be made to the illustrative embodiments and that other arrangements may be devised without departing from the spirit and scope of the present disclosure. The scope of the following claims is to be accorded the broadest interpretation so as to encompass all such modifications, equivalent structures, and functions.

1. A catheter comprising:
a braided inner cover or shaft;
a capture piece attached to a distal end of the braided inner cover or shaft so that the capture piece operates to capture or fully capture the braided inner cover or shaft or so that the capture piece operates to allow an end or ends of the braided inner cover or shaft to be secured and anchored; and
an atraumatic tip piece attached to or with a distal portion of the capture piece.

2. The catheter of claim 1, wherein one or more of the following:
(i) the atraumatic tip piece operates to bridge the capture piece to the braided inner cover or shaft;
(ii) the atraumatic tip piece includes a counterbore that operates to accommodate or bridge the capture piece and the braided inner cover or shaft;
(iii) the atraumatic tip piece is thermally attached to the distal portion of the catheter and/or to the distal portion of the capture piece and/or the braided inner cover or shaft;
(iv) the capture piece is made of higher durometer than the atraumatic piece so that the higher durometer operates to provide useful structure for the point and to preserve ends of the captured or fully captured braided inner cover or shaft; and/or
(v) the atraumatic tip piece has a durometer of one of the following: 35 D, 55 D, 63 D, 72 D, and/or a value in a range of 35 D-72 D.

3. The catheter of claim 1, wherein:
(i) the atraumatic tip piece includes a tool channel; and
(ii) the tool channel is connected to an inner diameter of the braided inner cover or shaft, and the connection is one or more of the following: continuous; connected so that the tool channel is co-axial with the inner diameter of the braided inner cover or shaft; in alignment; connected so that a diameter of the tool channel matches or substantially matches the inner diameter of the braided inner cover or shaft; and/or concatenated to create a concatenated channel that provides access to one or more imaging tools and/or medical tools from a proximal end of the catheter to a distal end of the catheter.

4. The catheter of claim 1, wherein one or more of the following:
(i) the capture piece and/or the atraumatic tip piece is/are made of polymer or a reflowable polymer, and/or Pebax or any other block copolymer variation of polyether block amide;
(ii) the capture piece and/or the atraumatic tip piece is/are made of thermoplastic elastomer; and/or
(iii) the capture piece and/or the atraumatic tip piece is/are made of a radiopaque material.

5. The catheter of claim 1, further comprising:
one or more drive or guide rings attached to the braided inner cover or shaft, the one or more drive or guide rings having one or more lumens or acceptors therein; and
one or more driving wires that are passively threaded through a corresponding lumen or acceptor of the one or more lumens or acceptors, the one or more driving wires being connected or terminated at a distal most drive or guide ring of the one or more drive or guide rings,
wherein one or more of the following: the distal most drive or guide ring is attached, connected to, or disposed near the atraumatic tip piece, the distal most drive or guide ring is attached, connected to, or disposed near the atraumatic tip piece such that the distal most drive or guide ring is flush with the atraumatic tip piece, and/or the one or more drive or guide rings are bonded on the braided inner cover or shaft with equidistant intervals between each drive or guide rings of the one or more drive or guide rings.

6. The catheter of claim 5, wherein the atraumatic tip piece includes one or more extruded bosses, wherein the one or more extruded bosses are inserted into and/or connected to a corresponding lumen or acceptor in the distalmost drive or guide ring, and/or wherein the one or more extruded bosses are keyed or attached to multi-lumens on the distalmost drive or guide ring and/or on a drive ring anchor or anchors.

7. The catheter of claim 6, wherein the atraumatic tip further includes a tool channel, the tool channel of the atraumatic tip operating to include an orientation feature or structure and to include a camera that is detachably attached with or in the tool channel.

8. The catheter of claim 7, wherein the orientation feature operates to determine rotational orientation, position, or state of a camera, an imaging device, and/or a medical tool disposed in the catheter with respect to the atraumatic tip piece and/or to suppress uncontrolled rotation or movement between the camera and the atraumatic tip piece, and the one or more extruded bosses operate to determine rotational orientation, position, or state of the atraumatic tip to or with respect to the distal most drive or guide ring.

9. The catheter of claim 5, wherein one or more of the following:
(i) the one or more driving wires operate to bend the catheter by pulling and pushing the one or more driving wire or wires;
(ii) the catheter has multiple bending sections and one or more processors that operate to control or command the multiple bending sections of the catheter using one or more of the following modes: a Follow the Leader (FTL) mode, a Reverse Follow the Leader (RFTL) mode, a Hold the Line mode, a Close the Gap mode, and/or a Stay the Course mode;
(iii) the catheter has multiple bending sections and one or more processors that operate to control movement of the catheter and/or to control a medical device or tool or an imaging device or tool to advance into, and/or retract from within, a tool channel of the catheter; and/or
(iv) the catheter is part of, includes, or is attached to an imaging device or system, an endoscope, a bronchoscope, a continuum robot, and/or an apparatus or system that operates to steer the catheter.

10. The catheter of claim 9, wherein, in a case where the catheter includes the one or more processors, one or more of the following features are part of the catheter:
the one or more processors further operate to use a neural network, convolutional neural network, or other AI-based method or feature and classify a pixel of an image or images obtained or received to a tissue type;
the one or more processors further operate to display results of the tissue classification completion on a display, store the results in a memory, or use the results to train one or more models or AI-networks to auto-detect or auto-characterize the tissue type; and/or
in a case where the one or more processors train one or more models or AI-networks, the one or more trained models or AI-networks is or uses one or a combination of the following: a neural net model or neural network model, a deep convolutional neural network model, a recurrent neural network model with long short-term memory that can take temporal relationships across images or frames into account, a generative adversarial network (GAN) model, a consistent generative adversarial network (cGAN) model, a three cycle-consistent generative adversarial network (3cGAN) model, a model that can take temporal relationships across images or frames into account, a model that can take temporal relationships into account including tissue location(s) during pullback in a vessel and/or including tissue characterization data during pullback in a vessel, a model that can use prior knowledge about a procedure and incorporate the prior knowledge into the machine learning algorithm or a loss function, a model using feature pyramid(s) that can take different image resolutions into account, and/or a model using residual learning technique(s); a segmentation model, a segmentation model with post-processing, a model with pre-processing, a model with post-processing, a segmentation model with pre-processing, a deep learning or machine learning model, a semantic segmentation model or classification model, an object detection or regression model, an object detection or regression model with pre-processing or post-processing, a combination of a semantic segmentation model and an object detection or regression model, a model using repeated segmentation model technique(s), a model using feature pyramid(s), a genetic algorithm that operates to breed multiple models for improved performance, and/or a model using repeated object detection or regression model technique(s).

11. The catheter of claim 1, further comprising a tracking sensor that operates to measure a position, orientation, and/or state of a distal end of the catheter, wherein the tracking sensor is attached to or in the atraumatic tip piece, and/or wherein the tracking sensor operates to locate an area from a distal end of the atraumatic tip to the capture piece along a longitudinal direction or along a longitudinal axis of the catheter.

12. The catheter of claim 1, further comprising one or more of RF stripes, radiopaque stripes, or other alternative color stripes that operate to one or more of the following: (i) indicate an orientation, position, or state of the atraumatic tip and capture during an attachment of the atraumatic tip to the capture piece and/or the braided inner cover or shaft, and/or (ii) aid in camera loading and docking into an anti-twist feature on a cap of the atraumatic tip.

13. A method for controlling a catheter having a braided inner cover or shaft, a capture piece attached to a distal end of the braided inner cover or shaft so that the capture piece operates to capture or fully capture the braided inner cover or shaft or so that the capture piece operates to allow an end or ends of the braided inner cover or shaft to be secured and anchored, and an atraumatic tip piece attached to or with a distal portion of the capture piece, the method comprising:
bending a distal portion of the catheter; and
advancing the catheter through a pathway,
wherein the catheter is advanced through the pathway in a substantially centered manner.

14. The method of claim 13, wherein one or more of the following:
(i) the atraumatic tip piece operates to bridge the capture piece to the braided inner cover or shaft;
(ii) the atraumatic tip piece includes a counterbore that operates to accommodate or bridge the capture piece and the braided inner cover or shaft;
(iii) the atraumatic tip piece is thermally attached to the distal portion of the catheter and/or to the distal portion of the capture piece and/or the braided inner cover or shaft;
(iv) the capture piece is made of higher durometer than the atraumatic piece so that the higher durometer operates to provide useful structure for the point and to preserve ends of the captured or fully captured braided inner cover or shaft; and/or
(v) the atraumatic tip piece has a durometer of one of the following: 35 D, 55 D, 63 D, 72 D, and/or a value in a range of 35 D-72 D.

15. The method of claim 13, wherein:
(i) the atraumatic tip piece includes a tool channel; and
(ii) the tool channel is connected to an inner diameter of the braided inner cover or shaft, and the connection is one or more of the following: continuous; connected so that the tool channel is co-axial with the inner diameter of the braided inner cover or shaft; in alignment; connected so that a diameter of the tool channel matches or substantially matches the inner diameter of the braided inner cover or shaft; and/or concatenated to create a concatenated channel that provides access to one or more imaging tools and/or medical tools from a proximal end of the catheter to a distal end of the catheter.

16. The method of claim 13, wherein the catheter further comprises:
one or more drive or guide rings attached to the braided inner cover or shaft, the one or more drive or guide rings having one or more lumens or acceptors therein; and
one or more driving wires that are passively threaded through a corresponding lumen or acceptor of the one or more lumens or acceptors, the one or more driving wires being connected or terminated at a distal most drive or guide ring of the one or more drive or guide rings, and
wherein one or more of the following: the distal most drive or guide ring is attached, connected to, or disposed near the atraumatic tip piece, the distal most drive or guide ring is attached, connected to, or disposed near the atraumatic tip piece such that the distal most drive or guide ring is flush with the atraumatic tip piece, and/or the one or more drive or guide rings are bonded on the braided inner cover or shaft with equidistant intervals between each drive or guide rings of the one or more drive or guide rings.

17. The method of claim 16, wherein the atraumatic tip piece includes one or more extruded bosses, wherein the one or more extruded bosses are inserted into and/or connected to a corresponding lumen or acceptor in the distalmost drive or guide ring, and/or wherein the one or more extruded bosses are keyed or attached to multi-lumens on the distalmost drive or guide ring and/or on a drive ring anchor or anchors.

18. The method of claim 17, wherein the atraumatic tip further includes a tool channel, the tool channel of the atraumatic tip operating to include an orientation feature or structure and to include a camera that is detachably attached with or in the tool channel.

19. The method of claim 18, further comprising using the orientation feature to determine rotational orientation, position, or state of a camera, an imaging device, and/or a medical tool disposed in the catheter with respect to the atraumatic tip piece and/or to suppress uncontrolled rotation or movement between the camera and the atraumatic tip piece, and using the one or more extruded bosses to determine rotational orientation, position, or state of the atraumatic tip to or with respect to the distal most drive or guide ring.

20. The method of claim 16, further comprising one or more of the following:
(i) using the one or more driving wires to bend the catheter by pulling and pushing the one or more driving wire or wires;
(ii) controlling or commanding multiple bending sections of the catheter to use one or more of the following modes: a Follow the Leader (FTL) mode, a Reverse Follow the Leader (RFTL) mode, a Hold the Line mode, a Close the Gap mode, and/or a Stay the Course mode;
(iii) controlling or commanding multiple bending sections of the catheter to control movement of the catheter and/or to control a medical device or tool or an imaging device or tool to advance into, and/or retract from within, a tool channel of the catheter; and/or
(iv) using the catheter as part of an imaging device or system, an endoscope, a bronchoscope, a continuum robot, and/or an apparatus or system that operates to steer the catheter.

21. The method of claim 20, further comprising:
using one or more processors and/or using a neural network, convolutional neural network, or other AI-based method or feature and classify a pixel of an image or images obtained or received to a tissue type;
using one or more processors to display results of the tissue classification completion on a display, store the results in a memory, or use the results to train one or more models or AI-networks to auto-detect or auto-characterize the tissue type; and/or
using one or more processors to train one or more models or AI-networks, and using the one or more trained models or AI-networks as or with one or a combination of the following: a neural net model or neural network model, a deep convolutional neural network model, a recurrent neural network model with long short-term memory that can take temporal relationships across images or frames into account, a generative adversarial network (GAN) model, a consistent generative adversarial network (cGAN) model, a three cycle-consistent generative adversarial network (3cGAN) model, a model that can take temporal relationships across images or frames into account, a model that can take temporal relationships into account including tissue location(s) during pullback in a vessel and/or including tissue characterization data during pullback in a vessel, a model that can use prior knowledge about a procedure and incorporate the prior knowledge into the machine learning algorithm or a loss function, a model using feature pyramid(s) that can take different image resolutions into account, and/or a model using residual learning technique(s); a segmentation model, a segmentation model with post-processing, a model with pre-processing, a model with post-processing, a segmentation model with pre-processing, a deep learning or machine learning model, a semantic segmentation model or classification model, an object detection or regression model, an object detection or regression model with pre-processing or post-processing, a combination of a semantic segmentation model and an object detection or regression model, a model using repeated segmentation model technique(s), a model using feature pyramid(s), a genetic algorithm that operates to breed multiple models for improved performance, and/or a model using repeated object detection or regression model technique(s).

22. The method of claim 13, further comprising using a tracking sensor in the catheter to one or more of the following: measure a position, orientation, and/or state of a distal end of the catheter, attaching the tracking sensor to or in the atraumatic tip piece, and/or using the tracking sensor to locate an area from a distal end of the atraumatic tip to the capture piece along a longitudinal direction or along a longitudinal axis of the catheter.

23. The method of claim 13, further comprising using one or more of RF stripes, radiopaque stripes, or other alternative color stripes on or for the catheter to one or more of the following: (i) indicate an orientation, position, or state of the atraumatic tip and capture during an attachment of the atraumatic tip to the capture piece and/or the braided inner cover or shaft, and/or (ii) aid in camera loading and docking into an anti-twist feature on a cap of the atraumatic tip.

24. A method of making a robotic catheter or an imaging apparatus, the method comprising:
attaching a distal end of a braided inner cover or shaft of a catheter with or to a capture piece; and
attaching an atraumatic tip piece to a distal end of the capture piece.

25. The method of claim 24, wherein one or more of the following:
(i) the attachment steps include or involve a thermal attachment process where at least the atraumatic tip piece is thermally attached to the catheter; and/or
(ii) the thermal attachment process provides: a stronger overall joint than adhesive processes, a continuous inner lumen of the catheter, and/or all free wire ends of the braided inner cover or shaft being fully captured and seated within reflowed polymer.

26. The method of claim 24, further comprising:
selecting a softer durometer material for a molded atraumatic tip piece as compared to the hardness of the capture piece to provide a configuration, wherein:
(i) the capture piece on the braided inner cover or shaft remains intact while the molded atraumatic tip piece is seated around the capture piece;
(ii) free wire ends of the braided inner cover or shaft do not penetrate, or avoid penetration with, an inner or outer diameter of the catheter; and
(iii) the wire ends are securely disposed or placed within a joint or bridge of the atraumatic tip piece that extends over and surrounds the capture piece of the catheter.

27. The method of claim 24, further comprising: applying or including radiopaque stripes, RF stripes, or other color stripes as visual aids on the catheter.

28. The method of claim 24, further comprising: using a specific, set, or predetermined internal geometry and/or structure for the molded piece for the atraumatic tip piece so that the catheter remains intact, and the specific, set, or predetermined internal geometry and/or structure of the catheter includes one or more of the following: (i) geometry and/or structure that operates to maintain or improve flow and vacuum suction rates; and/or (ii) geometry and/or structure that operates to achieve docking and movement of a camera within the catheter.

29. The method of claim 24, further comprising one or more of the following:
(i) using or adding extruded or extruding bosses on a proximal side of the atraumatic tip piece to align to a proximal skeleton or structure of the catheter;
(ii) disposing or incorporating a tracking sensor or electromagnetic (EM) sensor within geometry and/or structure of the atraumatic tip piece, or fully integrating the tracking sensor or EM sensor into the atraumatic tip to better maintain or to achieve a smooth outer diameter on the catheter.

30. A non-transitory computer-readable storage medium storing at least one program for causing a computer to execute a method for manufacturing a catheter, the method comprising:
attaching a distal end of a braided inner cover or shaft of a catheter with or to a capture piece; and
attaching an atraumatic tip piece to a distal end of the capture piece.
